(19) United States
(12) Patent Application Publication
(10) Pub. No.: US 2026/0327687 A1
(43) Pub. Date: Oct. 22, 2026

(54) ABSOLUTE QUANTIFICATION OF PROTEIN EXPRESSION AT SINGLE CELL LEVEL

(71) Applicant: Becton, Dickinson and Company, Franklin Lakes, NJ (US)

(72) Inventors: Jody Martin, San Jose, CA (US); Hye-Won Song, San Jose, CA (US); Joseph Ryan Olives, San Diego, CA (US)

(21) Appl. No.: 19/183,283

(22) Filed: Apr. 18, 2025

(51) Int. Cl. C12Q 1/6816; C12Q 1/6834; C12Q 1/6874; C40B 70/00

(52) U.S. Cl. CPC C12Q 1/6816; C12Q 1/6834; C12Q 1/6874; C40B 70/00

(57) ABSTRACT

Disclosed herein include systems, methods, compositions, and kits for the absolute quantification of protein expression at the single cell level. Quantification compositions are provided comprising a first particle population comprising two or more first particle subpopulations. Each first particle of a first particle subpopulation can comprise a predetermined number of copies of a quantification molecule. The two or more first particle subpopulations can differ with respect to the predetermined number of copies of the quantification molecule. Methods provided herein can comprise generating a correction factor by comparing the experimental number of copies of the quantification molecule in each first particle subpopulation with the predetermined number of copies of the quantification molecule in each first particle subpopulation.

### BACKGROUND

### Field

The present disclosure relates generally to the field of molecular biology, for example determining gene expression using molecular barcoding.

### Description of the Related Art

Current inefficiencies and experimental variables within CITE-seq workflows, such as, for example, inefficiency in oligonucleotide capture within the single cell platform workflow, unknown noise from unbound antibody-oligonucleotides in the system, and some number of uncounted molecules with sub-saturated sequencing reads, can yield a relative quantification of the protein targets on each individual cell rather than an absolute quantification. There is a need for methods, systems, compositions, and kits controlling for the inefficiencies and experimental variables within CITE-seq workflows to provide an absolute quantification of antigen density at the single cell level.

### SUMMARY

Disclosed herein include quantification compositions. In some embodiments, the quantification composition comprises: a first particle population comprising two or more first particle subpopulations, and a plurality of quantification molecule-binding reagents. In some embodiments, each first particle of a first particle subpopulation comprises a predetermined number of copies of a quantification molecule. In some embodiments, the two or more first particle subpopulations differ with respect to the predetermined number of copies of the quantification molecule. In some embodiments, each of the plurality of quantification molecule-binding reagents comprises a quantification molecule-binding reagent specific oligonucleotide comprising a unique identifier sequence for the quantification molecule. In some embodiments, the quantification molecule-binding reagent is capable of specifically binding to the quantification molecule.

Disclosed herein include quantification compositions. In some embodiments, the quantification composition comprises: a first particle population comprising two or more first particle subpopulations. In some embodiments, each first particle of a first particle subpopulation comprises a predetermined number of copies of a quantification molecule. In some embodiments, the two or more first particle subpopulations differ with respect to the predetermined number of copies of the quantification molecule.

In some embodiments, the quantification molecule comprises a protein (e.g., an intracellular protein, an extracellular protein, a cell-surface protein, a cell marker, a B-cell receptor, a T-cell receptor, a major histocompatibility complex, a tumor antigen, a receptor, or any combination thereof). In some embodiments, the quantification molecule comprises a carbohydrate or a lipid. In some embodiments, the quantification molecule comprises a detectable moiety (e.g., phycoerythrin (PE)). In some embodiments, the detectable moiety comprises an optical moiety, a luminescent moiety, a magnetic moiety, an electrochemically active moiety, a nanoparticle, or a combination thereof. In some embodiments, the luminescent moiety comprises a chemiluminescent moiety, an electroluminescent moiety, a photoluminescent moiety, or a combination thereof. some embodiments, the photoluminescent moiety comprises a fluorescent moiety, a phosphorescent moiety, or a combination thereof. In some embodiments, the fluorescent moiety comprises a fluorescent dye or a fluorescent protein. In some embodiments, the quantification molecule comprises a fluorescent protein selected from the group comprising Sirius, EBFP2, Azurite, mTagBFP2, mKalama1, TagBFP, Aquamarine, SCFP3A, mTurquoise, mTurquoise2, ECFP, Cerulean, mCerulean3, CyPet, TagCFP, AmCyan, mTFP1, mMidorishi-Cyan, mUKG, TurboGFP, Verdi GFP, Azami Green, ZsGreen, ZsGreen1, TagGFP2, hrGFP, hrGFP2, EGFP, Emerald, m Wasabi, Superfolder GFP, Clover, mNeonGreen, Monster Green, Renilla GFP, TurboYFP, Tag YFP, EYFP, Topaz, SYFP2, Venus, Citrine, YPet, mPapaya1, mKO, E2-Orange, mOrange, mOrange2, mKOk, mKO2, mNectarine, dTomato, tdTomato, DsRed, DsRed2, DsRed-Express, TagRFP, TagRFP-T, mTangerine, DsRed monomer, mApple, mStrawberry, mRuby2, mRuby, Fusion Red, mCherry, mLumin, mRaspberry, TagFP635 (mKate), mKate2, TurboFP635 (Katushka), HcRed-Tandem, mPlum, eqFP650, mNeptune, mCardinal, NirFP (eqFP670), TagRFP675, E2-Crimson, TagRFP657, BphP1-FP, iRFP670, iRFP713/V256C, iRFP682, iRFP702, IFP1.4, IFP2.0, iRFP713 (iRFP), iRFP720, T-Sapphire, mAmetrine, LSS-mOrange, LSS-mKate2, mBeRFP, mKeima Red, LSS-mKate1, or any combination thereof.

In some embodiments, the quantification molecules are present on the surface of the first particle. In some embodiments, the quantification molecules present on the surface of the first particle are conjugated by: (i) a 1,3-dipolar cycloaddition reaction, a hetero-Diels-Alder reaction, a nucleophilic substitution reaction, a non-aldol type carbonyl reaction, an addition to carbon-carbon multiple bond, an oxidation reaction, a click reaction, or any combination thereof; (ii) a reaction between acetylene and azide; and/or (iii) a reaction between an aldehyde or ketone group and a hydrazine or alkoxy amine. In some embodiments, the quantification molecule is absorption bound or covalently bound to the first particle. In some embodiments, the quantification molecule is associated with the first particle via non-covalent interactions (e.g., one or more of ionic bonding, hydrophobic interactions, van der Waals forces, and hydrogen bonding). In some embodiments, the quantification molecule is conjugated to the first particle by a 1,3-dipolar cycloaddition reaction, a hetero-Diels-Alder reaction, a nucleophilic substitution reaction, a non-aldol type carbonyl reaction, an addition to carbon-carbon multiple bond, an oxidation reaction, a click reaction, or any combination thereof. In some embodiments, the quantification molecule comprises a first ligand and the first particle comprises a second ligand, wherein the first ligand and the second ligand are a specific binding pair, wherein the specific binding pair comprises a first member of a specific binding pair and a second member of a specific binding pair that bind one another with high affinity, high avidity, and/or high specificity. In some embodiments, the binding between the first and second member of the specific binding pair occurs via covalent bonding; the binding between the first and second member of the specific binding pair occurs via non-covalent interactions; the binding between the first and the second member of the specific binding pair has a dissociation constant Ka between about 10⁻¹⁰ to about 10⁻¹⁵ mol/L; and/or the first and the second member of the specific binding pair is each selected from the group comprising an antibody or an antigen-binding portion thereof and an antigen, an biotin moiety and an avidin moiety, a dinitrophenol (DNP) and an anti-DNP antibody, a digoxin and an anti-digoxin antibody, a digoxigenin and an anti-digoxigenin antibody, a hapten and an anti-hapten, a polysaccharide and a polysaccharide binding moiety, a lectin and a receptor, a ligand and a receptor, a fluorescein and an anti-fluorescein antibody, complementary nucleic acids, derivatives therefore, and fragments thereof.

In some embodiments, the first particle comprises a bead. In some embodiments, the bead comprises: a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof; a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof; and/or a disruptable hydrogel particle. In some embodiments, the first particle comprises a diameter of about 10 μm to about 100 μm (e.g., about 35 μm). In some embodiments, the first particle is configured to have the dimensions of a mammalian cell (e.g., a human cell).

In some embodiments, the first particle population comprises at least 2, 3, 4, 5, 6, 7, 8, 9, or 10 first particle subpopulations. In some embodiments, each first particle subpopulation comprises at least about 2, 5, 10, 50, 100, 200, 500, 750, 1000, 2500, 5000, 7500, or 10000, first particles. In some embodiments, a first particle of a first particle subpopulation comprises at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, or 10000, quantification molecules. In some embodiments, the quantification molecule-binding reagent specific oligonucleotide comprises (i) a second molecular label and/or (ii) a second universal sequence. In some embodiments, at least ten of the plurality of quantification molecule-binding reagent specific oligonucleotides comprise different second molecular label sequences. In some embodiments, the quantification molecule-binding reagents comprise a protein, an antibody, an aptamer, a tetramer, a protein scaffold, or a combination thereof. In some embodiments, wherein the aptamer and the reagent oligonucleotide is a single polynucleotide. In some embodiments, the quantification molecule-binding reagents comprise an antibody or fragment thereof. In some embodiments, the antibody or fragment thereof comprises a monoclonal antibody, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, each first particle of a first particle subpopulation comprises a predetermined number of copies of one or more secondary quantification molecules. In some embodiments, the two or more first particle subpopulations differ with respect to the predetermined number of copies of said one or more secondary quantification molecules.

The quantification composition can comprise a second particle population comprising two or more second particle subpopulations. In some embodiments, each second particle of a second particle subpopulation comprises a predetermined number of copies of one or more secondary quantification molecules. In some embodiments, the two or more first particle subpopulations differ with respect to the predetermined number of copies of one or more secondary quantification molecules. The quantification composition can comprise a plurality of secondary quantification molecule-binding reagents. In some embodiments, each of the plurality of secondary quantification molecule-binding reagents comprises a secondary quantification molecule-binding reagent specific oligonucleotide comprising a unique identifier sequence for the secondary quantification molecule, and wherein the secondary quantification molecule-binding reagent is capable of specifically binding to at least one of the one or more secondary quantification molecules. In some embodiments, each first particle of a first particle subpopulation comprises first particle specific oligonucleotide(s) comprising a unique identifier sequence for the subpopulation. In some embodiments, at least one of the first particle specific oligonucleotide(s) is immobilized on, partially immobilized, enclosed in, or partially enclosed in the first particle. In some embodiments, the first particle specific oligonucleotide(s) is conjugated to the first particle. In some embodiments, the first particle specific oligonucleotide(s) is conjugated to the first particle through a chemical group selected from the group consisting of a UV photocleavable group, a streptavidin, a biotin, an amine, and a combination thereof. In some embodiments, the first particle specific oligonucleotide(s) comprise a sequence complementary to a capture sequence configured to capture the first particle specific oligonucleotide. In some embodiments, the sequence complementary to the capture sequence comprises a poly(dA) region. In some embodiments, each of the first particle specific oligonucleotide(s) is 10 to 500 nucleotides in length. In some embodiments, each of the first particle specific oligonucleotide(s) comprises a linker functional group. In some embodiments, the first particle comprises a solid support functional group. In some embodiments, the support functional group and the linker functional group are associated with each other. In some embodiments, the linker functional group and the support functional group are individually selected from the group consisting of C6, biotin, streptavidin, primary amine(s), aldehyde(s), ketone(s), and any combination thereof.

Disclosed herein include methods for cellular component quantification. In some embodiments, the method comprises: contacting one or more cells with a plurality of cellular component-binding reagents, wherein the one or more cells comprises a plurality of cellular component targets, wherein each of the plurality of cellular component-binding reagents comprises a cellular component-binding reagent specific oligonucleotide comprising a unique identifier sequence for the cellular component-binding reagent, and wherein the cellular component-binding reagent is capable of specifically binding to at least one of the plurality of cellular component targets. The method can comprise contacting a quantification composition disclosed herein with a plurality of quantification molecule-binding reagents, wherein each of the plurality of quantification molecule-binding reagents comprises a quantification molecule-binding reagent specific oligonucleotide comprising a unique identifier sequence for the quantification molecule, and wherein the quantification molecule-binding reagent is capable of specifically binding to the quantification molecule. The method can comprise barcoding the cellular component-binding reagent specific oligonucleotides with a plurality of oligonucleotide barcodes to generate a plurality of barcoded cellular component-binding reagent specific oligonucleotides each comprising a sequence complementary to at least a portion of the unique identifier sequence and a molecular label sequence. The method can comprise barcoding the quantification molecule-binding reagent specific oligonucleotides with a plurality of oligonucleotide barcodes to generate a plurality of barcoded quantification molecule-binding reagent specific oligonucleotides each comprising a sequence complementary to at least a portion of the unique identifier sequence and a molecular label sequence. The method can comprise obtaining sequencing data comprising a plurality of sequencing reads of the plurality of barcoded cellular component-binding reagent specific oligonucleotides, or products thereof, wherein each of the plurality of sequencing reads comprises a molecular label sequence and at least a portion of the unique identifier sequence. In some embodiments, the number of unique molecular label sequences associated with the unique identifier sequence for the cellular component-binding reagent capable of specifically binding to the at least one cellular component target in the sequencing data indicates a pre-adjusted number of copies of the at least one cellular component target in the one or more cells. The method can comprise obtaining sequencing data comprising a plurality of sequencing reads of the plurality of barcoded quantification molecule-binding reagent specific oligonucleotides, or products thereof, wherein each of the plurality of sequencing reads comprises a molecular label sequence and at least a portion of the unique identifier sequence. In some embodiments, the number of unique molecular label sequences associated with the unique identifier sequence for the quantification molecule-binding reagent in the sequencing data indicates the experimental number of copies of the quantification molecule in each first particle subpopulation. The method can comprise generating a correction factor by comparing the experimental number of copies of the quantification molecule in each first particle subpopulation with the predetermined number of copies of the quantification molecule in each first particle subpopulation. The method can comprise adjusting the pre-adjusted number of copies of the at least one cellular component target in the one or more cells with the correction factor to generate an adjusted number of copies of the at least one cellular component target in the one or more cells.

In some embodiments, generating the correction value comprises generating a calibration curve. In some embodiments, the method comprises contacting the quantification composition with the one or more cells, and wherein the step of contacting one or more cells with a plurality of cellular component-binding reagents comprises contacting the quantification composition with the plurality of quantification molecule-binding reagents. In some embodiments, the step of contacting one or more cells with the plurality of cellular component-binding reagents and the step of contacting the quantification composition with the plurality of quantification molecule-binding reagents are performed separately. In some embodiments, the plurality of cellular component-binding reagents comprises at least 10 cellular component-binding reagents each capable of specifically binding to at least one of at least 10 cellular component targets. In some embodiments, the cellular component target comprises an intracellular protein, a carbohydrate, a lipid, a protein, an extracellular protein, a cell marker, a B-cell receptor, a T-cell receptor, a major histocompatibility complex, a tumor antigen, a receptor, or any combination thereof.

In some embodiments, the at least one cellular component target does not comprise the quantification molecule or the one or more secondary quantification molecules. In some embodiments, the at least one cellular component target comprises the quantification molecule or the one or more secondary quantification molecules. In some embodiments, at least one of the quantification molecule-binding reagents and the cellular component-binding reagents are the same. In some embodiments, the quantification molecule-binding reagents and the cellular component-binding reagents comprise a protein, an antibody, an aptamer, a tetramer, a protein scaffold, or a combination thereof. In some embodiments, the aptamer and the reagent oligonucleotide is a single polynucleotide. In some embodiments, the quantification molecule-binding reagents and the cellular component-binding reagents comprise an antibody or fragment thereof. In some embodiments, the antibody or fragment thereof comprises a monoclonal antibody, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, the quantification molecule-binding reagents and cellular component-binding reagents have the same clonotype.

The method can comprise: after contacting the plurality of quantification molecule-binding reagents with the one or more cells, removing one or more quantification molecule-binding reagents of the plurality of quantification molecule-binding reagents that are not contacted with the first particle population. The method can comprise: removing the one or more quantification molecule-binding reagents not contacted with the first particle population comprises: removing the one or more quantification molecule-binding reagents not contacted with the quantification molecules. The method can comprise: after contacting the plurality of cellular component-binding reagents with the one or more cells, removing one or more cellular component-binding reagents of the plurality of cellular component-binding reagents that are not contacted with the one or more cells. In some embodiments, removing the one or more cellular component-binding reagents not contacted with the one or more cells comprises: removing the one or more cellular component-binding reagents not contacted with the respective at least one of the plurality of cellular component targets.

In some embodiments, the one or more cells comprise a plurality of single cells. In some embodiments, the plurality of single cells is derived from one or more samples (e.g., a plurality of samples). In some embodiments, the one or more cells comprises a plurality of cells, a plurality of single cells, a tissue, a tumor sample, or any combination thereof. In some embodiments, the sample comprises peripheral blood mononuclear cells or immune cells. In some embodiments, the immune cells comprises B cells, T cells or a combination thereof. In some embodiments, the contacting step comprises: partitioning the plurality of single cells, the first particle population, and a pool of solid supports to a plurality of partitions, wherein a partition of the plurality of partitions comprises a single cell and a single solid support, and wherein a partition of the plurality of partitions comprises a single first particle and a single solid support. In some embodiments, partition is a well or droplet. In some embodiments, the method comprises lysing each cell in the partitions. In some embodiments, the method comprises pooling the solid supports from said partitions.

In some embodiments, the plurality of oligonucleotide barcodes are associated with a solid support. In some embodiments, each oligonucleotide barcode comprises a first universal sequence, a first molecular label, and a target-binding region capable of hybridizing to one or more of a nucleic acid target, a cellular component-binding reagent specific oligonucleotide, a quantification molecule-binding reagent specific oligonucleotide, and a sample indexing oligonucleotide. In some embodiments, at least 10 of the plurality of oligonucleotide barcodes comprise different first molecular label sequences. In some embodiments, the plurality of oligonucleotide barcodes each comprise a cell label. In some embodiments, each cell label of the plurality of oligonucleotide barcodes comprises at least 6 nucleotides. In some embodiments, oligonucleotide barcodes associated with the same solid support comprise the same cell label. In some embodiments, oligonucleotide barcodes associated with different solid supports comprise different cell labels. In some embodiments, the cellular component-binding reagent specific oligonucleotide comprises one or more of: a second molecular label sequence; an alignment sequence adjacent to a poly(dA) region; a linker, wherein the cellular component-binding reagent specific oligonucleotide is associated with the first cellular component-binding reagent through the linker; a sequence complementary to a capture sequence configured to capture the cellular component-binding reagent specific oligonucleotide; and a second universal sequence. In some embodiments, at least ten of the plurality of cellular component-binding reagent specific oligonucleotides comprise different second molecular label sequences. In some embodiments, the second molecular label sequences of at least two cellular component-binding reagent specific oligonucleotides are different, and wherein the unique identifier sequences of the at least two cellular component-binding reagent specific oligonucleotides are identical. In some embodiments, the second molecular label sequences of at least two cellular component-binding reagent specific oligonucleotides are different, and wherein the unique identifier sequences of the at least two cellular component-binding reagent specific oligonucleotides are different. In some embodiments, the alignment sequence is one or more nucleotides, or two or more nucleotides, in length. In some embodiments, the sequence complementary to the capture sequence comprises a poly(dA) region.

The method can comprise: contacting the quantification composition disclosed herein with a plurality of secondary quantification molecule-binding reagents, wherein each of the plurality of secondary quantification molecule-binding reagents comprises a secondary quantification molecule-binding reagent specific oligonucleotide comprising a unique identifier sequence for the secondary quantification molecule, and wherein the secondary quantification molecule-binding reagent is capable of specifically binding to at least one of the one or more secondary quantification molecules. The method can comprise: barcoding the secondary quantification molecule-binding reagent specific oligonucleotides with a plurality of oligonucleotide barcodes to generate a plurality of barcoded secondary quantification molecule-binding reagent specific oligonucleotides each comprising a sequence complementary to at least a portion of the unique identifier sequence and a molecular label sequence. The method can comprise: obtaining sequencing data comprising a plurality of sequencing reads of the plurality of barcoded secondary quantification molecule-binding reagent specific oligonucleotides, or products thereof, wherein each of the plurality of sequencing reads comprises a molecular label sequence and at least a portion of the unique identifier sequence. In some embodiments, the number of unique molecular label sequences associated with the unique identifier sequence for the secondary quantification molecule-binding reagent in the sequencing data indicates the experimental number of copies of the secondary quantification molecule in each first particle subpopulation or second particle subpopulation.

In some embodiments, generating the correction factor comprises comparing the experimental number of copies of the secondary quantification molecule in each first particle subpopulation with the predetermined number of copies of the secondary quantification molecule in each first particle subpopulation. In some embodiments, generating the correction factor comprises comparing the experimental number of copies of the secondary quantification molecule in each second particle subpopulation with the predetermined number of copies of the secondary quantification molecule in each second particle subpopulation. In some embodiments, the number of unique molecular label sequences comprises the number of unique first molecular label sequences, the number of unique second molecular label sequences, or a combination thereof. In some embodiments, the method comprises: barcoding the first particle specific oligonucleotides with a plurality of oligonucleotide barcodes to generate a plurality of barcoded first particle specific oligonucleotides each comprising a sequence complementary to at least a portion of the unique identifier sequence and a molecular label sequence; obtaining sequencing data comprising a plurality of sequencing reads of the plurality of barcoded first particle specific oligonucleotides, or products thereof, wherein each of the plurality of sequencing reads comprises at least a portion of the unique identifier sequence and the cell label sequence; and associating the plurality of sequencing reads of the plurality of barcoded quantification molecule-binding reagent specific oligonucleotides, or products thereof, with a first particle subpopulation based on a shared cell label sequence.

In some embodiments, the one or more cells comprises copies of a nucleic acid target. The method can comprise: contacting the plurality of oligonucleotide barcodes with the copies of the nucleic acid target for hybridization; extending the plurality of oligonucleotide barcodes hybridized to the copies of a nucleic acid target to generate a plurality of barcoded nucleic acid molecules each comprising a sequence complementary to at least a portion of the nucleic acid target; and obtaining sequencing data of the plurality of barcoded nucleic acid molecules, or products thereof, to determine the copy number of the nucleic acid target in the one or more single cells. In some embodiments, extending the plurality of oligonucleotide barcodes hybridized to the copies of the nucleic acid target comprises extending the plurality of oligonucleotide barcodes hybridized to the copies of the nucleic acid target using a reverse transcriptase (e.g., a viral reverse transcriptase). In some embodiments, the method comprises determining the copy number of the nucleic acid target in the one or more cells based on the number of first molecular labels with distinct sequences associated with the plurality of barcoded nucleic acid molecules, or products thereof. In some embodiments, the sequence of the nucleic acid target in the plurality of barcoded nucleic acid molecules comprises a subsequence of the nucleic acid target. In some embodiments, determining the copy number of the nucleic acid target comprises determining the copy number of each of a plurality of nucleic acid targets in the one or more cells based on the number of molecular labels with distinct sequences associated with the plurality of barcoded nucleic acid molecules, or products thereof, comprising a sequence of the each of the plurality of nucleic acid targets. In some embodiments, the sequence of the each of the plurality of nucleic acid targets comprises a subsequence of the each of the plurality of nucleic acid targets.

Barcoding the cellular component-binding reagent specific oligonucleotides can comprise contacting with the plurality of oligonucleotide barcode for hybridization and extending the plurality of oligonucleotide barcodes hybridized to the cellular component-binding reagent specific oligonucleotides to generate the plurality of barcoded cellular component-binding reagent specific oligonucleotides. Barcoding the quantification molecule-binding reagent specific oligonucleotides can comprise contacting with the plurality of oligonucleotide barcode for hybridization and extending the plurality of oligonucleotide barcodes hybridized to the quantification molecule-binding reagent specific oligonucleotides to generate the plurality of barcoded quantification molecule-binding reagent specific oligonucleotides. Barcoding the secondary quantification molecule-binding reagent specific oligonucleotides can comprise contacting with the plurality of oligonucleotide barcode for hybridization and extending the plurality of oligonucleotide barcodes hybridized to the secondary quantification molecule-binding reagent specific oligonucleotides to generate the plurality of barcoded secondary quantification molecule-binding reagent specific oligonucleotides. Barcoding the first particle specific oligonucleotides can comprise contacting with the plurality of oligonucleotide barcode for hybridization and extending the plurality of oligonucleotide barcodes hybridized to the first particle specific oligonucleotides to generate the plurality of barcoded first particle specific oligonucleotides.

Barcoding the cellular component-binding reagent specific oligonucleotides can comprise contacting with the plurality of oligonucleotide barcode for hybridization and extending the cellular component-binding reagent specific oligonucleotides hybridized to the plurality of oligonucleotide barcodes to generate the plurality of barcoded cellular component-binding reagent specific oligonucleotides. Barcoding the quantification molecule-binding reagent specific oligonucleotides can comprise contacting with the plurality of oligonucleotide barcode for hybridization and extending the quantification molecule-binding reagent specific oligonucleotides hybridized to the plurality of oligonucleotide barcodes to generate the plurality of barcoded quantification molecule-binding reagent specific oligonucleotides. Barcoding the secondary quantification molecule-binding reagent specific oligonucleotides can comprise contacting with the plurality of oligonucleotide barcode for hybridization and extending the secondary quantification molecule-binding reagent specific oligonucleotides hybridized to the plurality of oligonucleotide barcodes to generate the plurality of barcoded secondary quantification molecule-binding reagent specific oligonucleotides. Barcoding the first particle specific oligonucleotides can comprise contacting with the plurality of oligonucleotide barcode for hybridization and extending the first particle specific oligonucleotides hybridized to the plurality of oligonucleotide barcodes to generate the plurality of barcoded first particle specific oligonucleotides. Obtaining sequencing data of the plurality of barcoded cellular component-binding reagent specific oligonucleotides, or products thereof, can comprise attaching the binding sites of sequencing primers and/or sequencing adaptors to the plurality of barcoded cellular component-binding reagent specific oligonucleotides, or products thereof.

Obtaining sequencing data of the plurality of barcoded quantification molecule-binding reagent specific oligonucleotides, or products thereof, can comprise attaching the binding sites of sequencing primers and/or sequencing adaptors to the plurality of barcoded quantification molecule-binding reagent specific oligonucleotides, or products thereof. Obtaining sequencing data of the plurality of barcoded secondary quantification molecule-binding reagent specific oligonucleotides, or products thereof, can comprise attaching the binding sites of sequencing primers and/or sequencing adaptors to the plurality of barcoded secondary quantification molecule-binding reagent specific oligonucleotides, or products thereof. Obtaining sequencing data of the plurality of barcoded first particle specific oligonucleotides, or products thereof, can comprise attaching the binding sites of sequencing primers and/or sequencing adaptors to the plurality of barcoded first particle specific oligonucleotides, or products thereof. Obtaining sequencing data of the plurality of barcoded nucleic acid molecules, or products thereof, can comprise attaching the binding sites of sequencing primers and/or sequencing adaptors to the plurality of barcoded nucleic acid molecules, or products thereof.

In some embodiments, the first universal sequence and the second universal sequence are the same. In some embodiments, the first universal sequence and the second universal sequence are different. In some embodiments, the first universal sequence and/or the second universal sequence comprise the binding sites of sequencing primers and/or sequencing adaptors, complementary sequences thereof, and/or portions thereof. In some embodiments, the sequencing adaptors comprise a P5 sequence, a P7 sequence, complementary sequences thereof, and/or portions thereof. In some embodiments, the sequencing primers comprise a Read 1 sequencing primer, a Read 2 sequencing primer, complementary sequences thereof, and/or portions thereof.

In some embodiments, the method comprises contacting each of the plurality of samples with a sample indexing composition of a plurality of sample indexing compositions, respectively. In some embodiments, each of the plurality of samples comprises one or more cells each comprising one or more cellular component targets, wherein the sample indexing composition comprises a cellular component-binding reagent associated with a sample indexing oligonucleotide, wherein the cellular component-binding reagent is capable of specifically binding to at least one of the one or more cellular component targets. In some embodiments, the sample indexing oligonucleotide comprises a sample indexing sequence, or a precursor thereof, and wherein sample indexing sequences of at least two sample indexing compositions of the plurality of sample indexing compositions comprise different sequences. The method can comprise: identifying sample origin of at least one cell of the one or more cells based on the sample indexing sequence of at least one sample indexing oligonucleotide of the plurality of sample indexing compositions. In some embodiments, identifying the sample origin of the at least one cell comprises: barcoding sample indexing oligonucleotides of the plurality of sample indexing compositions using the plurality of oligonucleotide barcodes to generate a plurality of barcoded sample indexing oligonucleotides; obtaining sequencing data of the plurality of barcoded sample indexing oligonucleotides, or products thereof; and identifying the sample origin of the cell based on the sample indexing sequence of at least one barcoded sample indexing oligonucleotide of the plurality of barcoded sample indexing oligonucleotides in the sequencing data. In some embodiments, identifying the sample origin of the at least one cell comprises identifying the presence or absence of the sample indexing sequence of at least one sample indexing oligonucleotide, or of at least one barcoded sample indexing oligonucleotide, in the sequencing data.

In some embodiments, the target-binding region comprises an oligo dT sequence, a random sequence, a target-specific sequence, or a combination thereof. In some embodiments, the target-binding region comprises a poly(dT) region, and wherein the nucleic acid target comprises a poly(dA) region. In some embodiments, the solid support comprises a synthetic particle. In some embodiments, the solid support comprises a planar surface. In some embodiments, at least one of the plurality of oligonucleotide barcodes is immobilized on, partially immobilized, enclosed in, or partially enclosed in the synthetic particle. In some embodiments, the synthetic particle is disruptable. In some embodiments, the synthetic particle comprises a bead. In some embodiments, the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof. In some embodiments, the synthetic particle comprises a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof. In some embodiments, the synthetic particle comprises a disruptable hydrogel particle. In some embodiments, each of the plurality of oligonucleotide barcodes comprises a linker functional group. In some embodiments, the synthetic particle comprises a solid support functional group. In some embodiments, the support functional group and the linker functional group are associated with each other. In some embodiments, the linker functional group and the support functional group are individually selected from the group consisting of C6, biotin, streptavidin, primary amine(s), aldehyde(s), ketone(s), and any combination thereof.

### BRIEF DESCRIPTION OF THE DRAWINGS

FIG. 1 illustrates a non-limiting exemplary stochastic barcode.

FIG. 2 shows a non-limiting exemplary workflow of stochastic barcoding and digital counting.

FIG. 3 is a schematic illustration showing a non-limiting exemplary process for generating an indexed library of the stochastically barcoded targets from a plurality of targets.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include quantification compositions. In some embodiments, the quantification composition comprises: a first particle population comprising two or more first particle subpopulations, and a plurality of quantification molecule-binding reagents. In some embodiments, each first particle of a first particle subpopulation comprises a predetermined number of copies of a quantification molecule. In some embodiments, the two or more first particle subpopulations differ with respect to the predetermined number of copies of the quantification molecule. In some embodiments, each of the plurality of quantification molecule-binding reagents comprises a quantification molecule-binding reagent specific oligonucleotide comprising a unique identifier sequence for the quantification molecule. In some embodiments, the quantification molecule-binding reagent is capable of specifically binding to the quantification molecule.

Disclosed herein include quantification compositions. In some embodiments, the quantification composition comprises: a first particle population comprising two or more first particle subpopulations. In some embodiments, each first particle of a first particle subpopulation comprises a predetermined number of copies of a quantification molecule. In some embodiments, the two or more first particle subpopulations differ with respect to the predetermined number of copies of the quantification molecule.

Disclosed herein include methods for cellular component quantification. In some embodiments, the method comprises: contacting one or more cells with a plurality of cellular component-binding reagents, wherein the one or more cells comprises a plurality of cellular component targets, wherein each of the plurality of cellular component-binding reagents comprises a cellular component-binding reagent specific oligonucleotide comprising a unique identifier sequence for the cellular component-binding reagent, and wherein the cellular component-binding reagent is capable of specifically binding to at least one of the plurality of cellular component targets. The method can comprise contacting a quantification composition disclosed herein with a plurality of quantification molecule-binding reagents, wherein each of the plurality of quantification molecule-binding reagents comprises a quantification molecule-binding reagent specific oligonucleotide comprising a unique identifier sequence for the quantification molecule, and wherein the quantification molecule-binding reagent is capable of specifically binding to the quantification molecule. The method can comprise barcoding the cellular component-binding reagent specific oligonucleotides with a plurality of oligonucleotide barcodes to generate a plurality of barcoded cellular component-binding reagent specific oligonucleotides each comprising a sequence complementary to at least a portion of the unique identifier sequence and a molecular label sequence. The method can comprise barcoding the quantification molecule-binding reagent specific oligonucleotides with a plurality of oligonucleotide barcodes to generate a plurality of barcoded quantification molecule-binding reagent specific oligonucleotides each comprising a sequence complementary to at least a portion of the unique identifier sequence and a molecular label sequence. The method can comprise obtaining sequencing data comprising a plurality of sequencing reads of the plurality of barcoded cellular component-binding reagent specific oligonucleotides, or products thereof, wherein each of the plurality of sequencing reads comprises a molecular label sequence and at least a portion of the unique identifier sequence. In some embodiments, the number of unique molecular label sequences associated with the unique identifier sequence for the cellular component-binding reagent capable of specifically binding to the at least one cellular component target in the sequencing data indicates a pre-adjusted number of copies of the at least one cellular component target in the one or more cells. The method can comprise obtaining sequencing data comprising a plurality of sequencing reads of the plurality of barcoded quantification molecule-binding reagent specific oligonucleotides, or products thereof, wherein each of the plurality of sequencing reads comprises a molecular label sequence and at least a portion of the unique identifier sequence. In some embodiments, the number of unique molecular label sequences associated with the unique identifier sequence for the quantification molecule-binding reagent in the sequencing data indicates the experimental number of copies of the quantification molecule in each first particle subpopulation. The method can comprise generating a correction factor by comparing the experimental number of copies of the quantification molecule in each first particle subpopulation with the predetermined number of copies of the quantification molecule in each first particle subpopulation. The method can comprise adjusting the pre-adjusted number of copies of the at least one cellular component target in the one or more cells with the correction factor to generate an adjusted number of copies of the at least one cellular component target in the one or more cells.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “adaptor” can mean a sequence to facilitate amplification or sequencing of associated nucleic acids. The associated nucleic acids can comprise target nucleic acids. The associated nucleic acids can comprise one or more of spatial labels, target labels, sample labels, indexing label, or barcode sequences (e.g., molecular labels). The adapters can be linear. The adaptors can be pre-adenylated adapters. The adaptors can be double-or single-stranded. One or more adaptor can be located on the 5′ or 3′ end of a nucleic acid. When the adaptors comprise known sequences on the 5′ and 3′ ends, the known sequences can be the same or different sequences. An adaptor located on the 5′ and/or 3′ ends of a polynucleotide can be capable of hybridizing to one or more oligonucleotides immobilized on a surface. An adapter can, in some embodiments, comprise a universal sequence. A universal sequence can be a region of nucleotide sequence that is common to two or more nucleic acid molecules. The two or more nucleic acid molecules can also have regions of different sequence. Thus, for example, the 5′ adapters can comprise identical and/or universal nucleic acid sequences and the 3′ adapters can comprise identical and/or universal sequences. A universal sequence that may be present in different members of a plurality of nucleic acid molecules can allow the replication or amplification of multiple different sequences using a single universal primer that is complementary to the universal sequence. Similarly, at least one, two (e.g., a pair) or more universal sequences that may be present in different members of a collection of nucleic acid molecules can allow the replication or amplification of multiple different sequences using at least one, two (e.g., a pair) or more single universal primers that are complementary to the universal sequences. Thus, a universal primer includes a sequence that can hybridize to such a universal sequence. The target nucleic acid sequence-bearing molecules may be modified to attach universal adapters (e.g., non-target nucleic acid sequences) to one or both ends of the different target nucleic acid sequences. The one or more universal primers attached to the target nucleic acid can provide sites for hybridization of universal primers. The one or more universal primers attached to the target nucleic acid can be the same or different from each other.

As used herein, an antibody can be a full-length (e.g., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

In some embodiments, an antibody is a functional antibody fragment. For example, an antibody fragment can be a portion of an antibody such as F(ab′)2, Fab′, Fab, Fv, sFv and the like. An antibody fragment can bind with the same antigen that is recognized by the full-length antibody. An antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (e.g., CD8, CD34, and CD45), and therapeutic antibodies.

As used herein the term “associated” or “associated with” can mean that two or more species are identifiable as being co-located at a point in time. An association can mean that two or more species are or were within a similar container. An association can be an informatics association. For example, digital information regarding two or more species can be stored and can be used to determine that one or more of the species were co-located at a point in time. An association can also be a physical association. In some embodiments, two or more associated species are “tethered”, “attached”, or “immobilized” to one another or to a common solid or semisolid surface. An association may refer to covalent or non-covalent means for attaching labels to solid or semi-solid supports such as beads. An association may be a covalent bond between a target and a label. An association can comprise hybridization between two molecules (such as a target molecule and a label).

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “digital counting” can refer to a method for estimating a number of target molecules in a sample. Digital counting can include the step of determining a number of unique labels that have been associated with targets in a sample. This methodology, which can be stochastic in nature, transforms the problem of counting molecules from one of locating and identifying identical molecules to a series of yes/no digital questions regarding detection of a set of predefined labels.

As used herein, the term “label” or “labels” can refer to nucleic acid codes associated with a target within a sample. A label can be, for example, a nucleic acid label. A label can be an entirely or partially amplifiable label. A label can be entirely or partially sequencable label. A label can be a portion of a native nucleic acid that is identifiable as distinct. A label can be a known sequence. A label can comprise a junction of nucleic acid sequences, for example a junction of a native and non-native sequence. As used herein, the term “label” can be used interchangeably with the terms, “index”, “tag,” or “label-tag.” Labels can convey information. For example, in various embodiments, labels can be used to determine an identity of a sample, a source of a sample, an identity of a cell, and/or a target.

As used herein, the term “non-depleting reservoirs” can refer to a pool of barcodes (e.g., stochastic barcodes) made up of many different labels. A non-depleting reservoir can comprise large numbers of different barcodes such that when the non-depleting reservoir is associated with a pool of targets each target is likely to be associated with a unique barcode. The uniqueness of each labeled target molecule can be determined by the statistics of random choice, and depends on the number of copies of identical target molecules in the collection compared to the diversity of labels. The size of the resulting set of labeled target molecules can be determined by the stochastic nature of the barcoding process, and analysis of the number of barcodes detected then allows calculation of the number of target molecules present in the original collection or sample. When the ratio of the number of copies of a target molecule present to the number of unique barcodes is low, the labeled target molecules are highly unique (i.e., there is a very low probability that more than one target molecule will have been labeled with a given label).

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. Linear compounds can have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (e.g., morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid can also include nucleobase (also referred to as “base”) modifications or substitutions. As used herein, “unmodified” or “natural” nucleobases can include the purine bases, (e.g., adenine (A) and guanine (G)), and the pyrimidine bases, (e.g., thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′: 4,5)pyrrolo[2,3-d]pyrimidin-2-one).

As used herein, the term “sample” can refer to a composition comprising targets. Suitable samples for analysis by the disclosed methods, devices, and systems include cells, tissues, organs, or organisms. As used herein, the term “sampling device” or “device” can refer to a device which may take a section of a sample and/or place the section on a substrate. A sample device can refer to, for example, a fluorescence activated cell sorting (FACS) machine, a cell sorter machine, a biopsy needle, a biopsy device, a tissue sectioning device, a microfluidic device, a blade grid, and/or a microtome.

As used herein, the term “solid support” can refer to discrete solid or semi-solid surfaces to which a plurality of barcodes (e.g., stochastic barcodes) may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A plurality of solid supports spaced in an array may not comprise a substrate. A solid support may be used interchangeably with the term “bead.”As used herein, the term “stochastic barcode” can refer to a polynucleotide sequence comprising labels of the present disclosure. A stochastic barcode can be a polynucleotide sequence that can be used for stochastic barcoding. Stochastic barcodes can be used to quantify targets within a sample. Stochastic barcodes can be used to control for errors which may occur after a label is associated with a target. For example, a stochastic barcode can be used to assess amplification or sequencing errors. A stochastic barcode associated with a target can be called a stochastic barcode-target or stochastic barcode-tag-target.

As used herein, the term “gene-specific stochastic barcode” can refer to a polynucleotide sequence comprising labels and a target-binding region that is gene-specific. A stochastic barcode can be a polynucleotide sequence that can be used for stochastic barcoding. Stochastic barcodes can be used to quantify targets within a sample. Stochastic barcodes can be used to control for errors which may occur after a label is associated with a target. For example, a stochastic barcode can be used to assess amplification or sequencing errors. A stochastic barcode associated with a target can be called a stochastic barcode-target or stochastic barcode-tag-target.

As used herein, the term “stochastic barcoding” can refer to the random labeling (e.g., barcoding) of nucleic acids. Stochastic barcoding can utilize a recursive Poisson strategy to associate and quantify labels associated with targets. As used herein, the term “stochastic barcoding” can be used interchangeably with “stochastic labeling.”

As used here, the term “target” can refer to a composition which can be associated with a barcode (e.g., a stochastic barcode). Exemplary suitable targets for analysis by the disclosed methods, devices, and systems include oligonucleotides, DNA, RNA, mRNA, microRNA, tRNA, and the like. Targets can be single or double stranded. In some embodiments, targets can be proteins, peptides, or polypeptides. In some embodiments, targets are lipids. As used herein, “target” can be used interchangeably with “species.”

As used herein, the term “reverse transcriptases” can refer to a group of enzymes having reverse transcriptase activity (i.e., that catalyze synthesis of DNA from an RNA template). In general, such enzymes include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants or derivatives thereof. Non-retroviral reverse transcriptases include non-LTR retrotransposon reverse transcriptases, retroplasmid reverse transcriptases, retron reverse transcriptases, and group II intron reverse transcriptases. Examples of group II intron reverse transcriptases include the Lactococcus lactis LI.LtrB intron reverse transcriptase, the Thermosynechococcus elongatus TeI4c intron reverse transcriptase, or the Geobacillus stearothermophilus GsI-IIC intron reverse transcriptase. Other classes of reverse transcriptases can include many classes of non-retroviral reverse transcriptases (i.e., retrons, group II introns, and diversity-generating retroelements among others).

The terms “universal adaptor primer,” “universal primer adaptor” or “universal adaptor sequence” are used interchangeably to refer to a nucleotide sequence that can be used to hybridize to barcodes (e.g., stochastic barcodes) to generate gene-specific barcodes. A universal adaptor sequence can, for example, be a known sequence that is universal across all barcodes used in methods of the disclosure. For example, when multiple targets are being labeled using the methods disclosed herein, each of the target-specific sequences may be linked to the same universal adaptor sequence. In some embodiments, more than one universal adaptor sequences may be used in the methods disclosed herein. For example, when multiple targets are being labeled using the methods disclosed herein, at least two of the target-specific sequences are linked to different universal adaptor sequences. A universal adaptor primer and its complement may be included in two oligonucleotides, one of which comprises a target-specific sequence and the other comprises a barcode. For example, a universal adaptor sequence may be part of an oligonucleotide comprising a target-specific sequence to generate a nucleotide sequence that is complementary to a target nucleic acid. A second oligonucleotide comprising a barcode and a complementary sequence of the universal adaptor sequence may hybridize with the nucleotide sequence and generate a target-specific barcode (e.g., a target-specific stochastic barcode). A universal adaptor primer can have a sequence that is different from a universal PCR primer used in the methods of this disclosure.

### Barcodes

Barcoding, such as stochastic barcoding, has been described in, for example, Fu et al., Proc Natl Acad Sci U.S.A., 2011 May 31,108(22): 9026-31; US 2011/0160078; Fan et al., Science, 2015, 347(6222): 1258367; US2015/0299784; and WO2015/031691; the content of each of these, including any supporting or supplemental information or material, is incorporated herein by reference in its entirety. In some embodiments, the barcode disclosed herein can be a stochastic barcode which can be a polynucleotide sequence that may be used to stochastically label (e.g., barcode, tag) a target. Barcodes can be referred to stochastic barcodes if the ratio of the number of different barcode sequences of the stochastic barcodes and the number of occurrence of any of the targets to be labeled can be, or be about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, 100:1, or a number or a range between any two of these values. A target can be an mRNA species comprising mRNA molecules with identical or nearly identical sequences. Barcodes can be referred to as stochastic barcodes if the ratio of the number of different barcode sequences of the stochastic barcodes and the number of occurrence of any of the targets to be labeled is at least, or is at most, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, or 100:1. Barcode sequences of stochastic barcodes can be referred to as molecular labels.

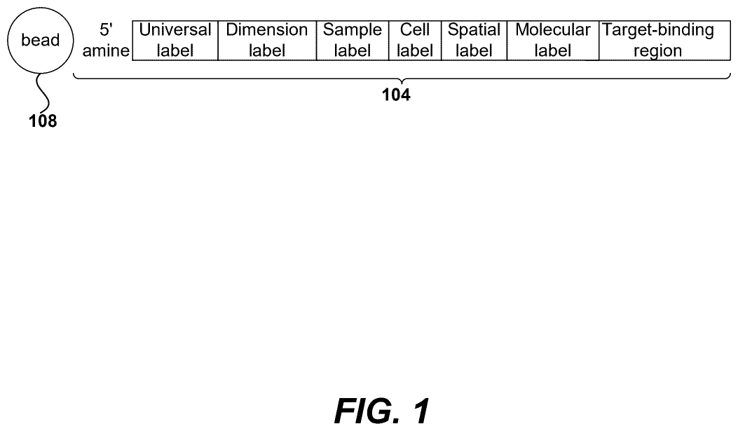

A barcode, for example a stochastic barcode, can comprise one or more labels. Exemplary labels can include a universal label, a cell label, a barcode sequence (e.g., a molecular label), a sample label, a plate label, a spatial label, and/or a pre-spatial label. FIG. 1 illustrates an exemplary barcode 104 with a spatial label. The barcode 104 can comprise a 5′ amine that may link the barcode to a solid support 108. The barcode can comprise a universal label, a dimension label, a spatial label, a cell label, and/or a molecular label. The order of different labels (including but not limited to the universal label, the dimension label, the spatial label, the cell label, and the molecule label) in the barcode can vary. For example, as shown in FIG. 1, the universal label may be the 5′-most label, and the molecular label may be the 3′-most label. The spatial label, dimension label, and the cell label can be in any order. In some embodiments, the universal label, the spatial label, the dimension label, the cell label, and the molecular label are in any order. The barcode can comprise a target-binding region. The target-binding region can interact with a target (e.g., target nucleic acid, RNA, mRNA, DNA) in a sample. For example, a target-binding region can comprise an oligo(dT) sequence which can interact with poly(A) tails of mRNAs. In some embodiments, the labels of the barcode (e.g., universal label, dimension label, spatial label, cell label, and barcode sequence) are separated by 1, 2, 3, 4, 5, 6, 7, 8, 9, 10, 11, 12, 13, 14, 15, 16, 17, 18, 19, 20, or more nucleotides.

A label, for example the cell label, can comprise a unique set of nucleic acid sub-sequences of defined length, e.g., seven nucleotides each (equivalent to the number of bits used in some Hamming error correction codes), which can be designed to provide error correction capability. The set of error correction sub-sequences comprise seven nucleotide sequences can be designed such that any pairwise combination of sequences in the set exhibits a defined “genetic distance” (or number of mismatched bases), for example, a set of error correction sub-sequences can be designed to exhibit a genetic distance of three nucleotides. In this case, review of the error correction sequences in the set of sequence data for labeled target nucleic acid molecules (described more fully below) can allow one to detect or correct amplification or sequencing errors. In some embodiments, the length of the nucleic acid sub-sequences used for creating error correction codes can vary, for example, they can be, or be about 1, 2, 3, 4, 5, 6, 7, 8, 9, 10, 15, 20, 30, 31, 40, 50, or a number or a range between any two of these values, nucleotides in length. In some embodiments, nucleic acid sub-sequences of other lengths can be used for creating error correction codes.

The barcode can comprise a target-binding region. The target-binding region can interact with a target in a sample. The target can be, or comprise, ribonucleic acids (RNAs), messenger RNAs (mRNAs), microRNAs, small interfering RNAs (siRNAs), RNA degradation products, RNAs each comprising a poly(A) tail, or any combination thereof. In some embodiments, the plurality of targets can include deoxyribonucleic acids (DNAs).

In some embodiments, a target-binding region can comprise an oligo(dT) sequence which can interact with poly(A) tails of mRNAs. One or more of the labels of the barcode (e.g., the universal label, the dimension label, the spatial label, the cell label, and the barcode sequences (e.g., molecular label)) can be separated by a spacer from another one or two of the remaining labels of the barcode. The spacer can be, for example, 1, 2, 3, 4, 5, 6, 7, 8, 9, 10, 11, 12, 13, 14, 15, 16, 17, 18, 19, or 20, or more nucleotides. In some embodiments, none of the labels of the barcode is separated by spacer.

### Universal Labels

A barcode can comprise one or more universal labels. In some embodiments, the one or more universal labels can be the same for all barcodes in the set of barcodes attached to a given solid support. In some embodiments, the one or more universal labels can be the same for all barcodes attached to a plurality of beads. In some embodiments, a universal label can comprise a nucleic acid sequence that is capable of hybridizing to a sequencing primer. Sequencing primers can be used for sequencing barcodes comprising a universal label. Sequencing primers (e.g., universal sequencing primers) can comprise sequencing primers associated with high-throughput sequencing platforms. In some embodiments, a universal label can comprise a nucleic acid sequence that is capable of hybridizing to a PCR primer. In some embodiments, the universal label can comprise a nucleic acid sequence that is capable of hybridizing to a sequencing primer and a PCR primer. The nucleic acid sequence of the universal label that is capable of hybridizing to a sequencing or PCR primer can be referred to as a primer binding site. A universal label can comprise a sequence that can be used to initiate transcription of the barcode. A universal label can comprise a sequence that can be used for extension of the barcode or a region within the barcode. A universal label can be, be about, be at least, or be at most, 1, 2, 3, 4, 5, 10, 15, 20, 25, 30, 35, 40, 45, 50, 100, 200, 300, or a number or a range between any two of these values, nucleotides in length. In some embodiments, a cleavable linker or modified nucleotide can be part of the universal label sequence to enable the barcode to be cleaved off from the support.

### Dimension Labels

A barcode can comprise one or more dimension labels. In some embodiments, a dimension label can comprise a nucleic acid sequence that provides information about a dimension in which the labeling (e.g., stochastic labeling) occurred. For example, a dimension label can provide information about the time at which a target was barcoded. A dimension label can be associated with a time of barcoding (e.g., stochastic barcoding) in a sample. A dimension label can be activated at the time of labeling. Different dimension labels can be activated at different times. The dimension label provides information about the order in which targets, groups of targets, and/or samples were barcoded. For example, a population of cells can be barcoded at the GO phase of the cell cycle. The cells can be pulsed again with barcodes (e.g., stochastic barcodes) at the G1 phase of the cell cycle. The cells can be pulsed again with barcodes at the S phase of the cell cycle, and so on. Barcodes at each pulse (e.g., each phase of the cell cycle), can comprise different dimension labels. In this way, the dimension label provides information about which targets were labelled at which phase of the cell cycle. Dimension labels can interrogate many different biological times. Exemplary biological times can include, but are not limited to, the cell cycle, transcription (e.g., transcription initiation), and transcript degradation. In another example, a sample (e.g., a cell, a population of cells) can be labeled before and/or after treatment with a drug and/or therapy. The changes in the number of copies of distinct targets can be indicative of the sample's response to the drug and/or therapy.

A dimension label can be activatable. An activatable dimension label can be activated at a specific time point. The activatable label can be, for example, constitutively activated (e.g., not turned off). The activatable dimension label can be, for example, reversibly activated (e.g., the activatable dimension label can be turned on and turned off). The dimension label can be, for example, reversibly activatable at least 1, 2, 3, 4, 5, 6, 7, 8, 9, 10, or more times. In some embodiments, the dimension label can be activated with fluorescence, light, a chemical event (e.g., cleavage, ligation of another molecule, addition of modifications (e.g., pegylated, sumoylated, acetylated, methylated, deacetylated, demethylated), a photochemical event (e.g., photocaging), and introduction of a non-natural nucleotide.

The dimension label can, in some embodiments, be identical for all barcodes (e.g., stochastic barcodes) attached to a given solid support (e.g., a bead), but different for different solid supports (e.g., beads). In some embodiments, at least 60%, 70%, 80%, 85%, 90%, 95%, 97%, 99% or 100%, of barcodes on the same solid support can comprise the same dimension label.

There can be as many as 106 or more unique dimension label sequences represented in a plurality of solid supports (e.g., beads). A dimension label can be, be about, be at least, or be at most, 1, 2, 3, 4, 5, 10, 15, 20, 25, 30, 35, 40, 45, 50, 75, 100, 125, 150, 200, or a number or a range between any two of these values, nucleotides in length.

### Spatial Labels

A barcode can comprise one or more spatial labels. In some embodiments, a spatial label can comprise a nucleic acid sequence that provides information about the spatial orientation of a target molecule which is associated with the barcode. A spatial label can be associated with a coordinate in a sample. The coordinate can be a fixed coordinate. For example, a coordinate can be fixed in reference to a substrate. A spatial label can be in reference to a two or three-dimensional grid. A coordinate can be fixed in reference to a landmark. The landmark can be identifiable in space. A landmark can be a structure which can be imaged. A landmark can be a biological structure, for example an anatomical landmark. A landmark can be a cellular landmark, for instance an organelle. A landmark can be a non-natural landmark such as a structure with an identifiable identifier such as a color code, bar code, magnetic property, fluorescents, radioactivity, or a unique size or shape. A spatial label can be associated with a physical partition (e.g., a well, a container, or a droplet). In some embodiments, multiple spatial labels are used together to encode one or more positions in space.

The spatial label can be identical for all barcodes attached to a given solid support (e.g., a bead), but different for different solid supports (e.g., beads). In some embodiments, the percentage of barcodes on the same solid support comprising the same spatial label can be, be about, be at least, or be at most, 60%, 70%, 80%, 85%, 90%, 95%, 97%, 99%, 100%, or a number or a range between any two of these values.

There can be as many as 106 or more unique spatial label sequences represented in a plurality of solid supports (e.g., beads). A spatial label can be, be about, at least or at most, 1, 2, 3, 4, 5, 10, 15, 20, 25, 30, 35, 40, 45, 50, 100, 125, 150, 175, 200, 250, or 300 or a number or a range between any two of these values, nucleotides in length.

### Cell Labels

A barcode (e.g., a stochastic barcode) can comprise one or more cell labels. In some embodiments, a cell label can comprise a nucleic acid sequence that provides information for determining which target nucleic acid originated from which cell. In some embodiments, the cell label is identical for all barcodes attached to a given solid support (e.g., a bead), but different for different solid supports (e.g., beads). In some embodiments, the percentage of barcodes on the same solid support comprising the same cell label can be, be about, at most, or be at least, 60%, 70%, 80%, 85%,90%, 95%, 97%, 99%, 100%, or a number or a range between any two of these values.

There can be as many as 106 or more unique cell label sequences represented in a plurality of solid supports (e.g., beads). A cell label can be, be about, at least, or be at most, 1, 2, 3, 4, 5, 10, 15, 20, 25, 30, 35, 40, 45, 50, 100, 125, 150, 175, 200, or a number or a range between any two of these values, nucleotides in length.

### Barcode Sequences

A barcode can comprise one or more barcode sequences. In some embodiments, a barcode sequence can comprise a nucleic acid sequence that provides identifying information for the specific type of target nucleic acid species hybridized to the barcode. A barcode sequence can comprise a nucleic acid sequence that provides a counter (e.g., that provides a rough approximation) for the specific occurrence of the target nucleic acid species hybridized to the barcode (e.g., target-binding region).

In some embodiments, a diverse set of barcode sequences are attached to a given solid support (e.g., a bead). In some embodiments, there can be, or be about, 10², 10³, 10⁴, 10⁵, 10⁶, 10⁷, 10⁸, 10⁹, or a number or a range between any two of these values, unique molecular label sequences. For example, a plurality of barcodes can comprise about 6561 barcodes sequences with distinct sequences. As another example, a plurality of barcodes can comprise about 65536 barcode sequences with distinct sequences. In some embodiments, there can be at least, or be at most, 10², 10³, 10⁴, 10⁵, 10⁶, 10⁷, 10⁸, or 10⁹, unique barcode sequences. The unique molecular label sequences can be attached to a given solid support (e.g., a bead).

The length of a barcode can be different in different implementations. For example, a barcode can be, be about, be at least, or be at most, 1, 2, 3, 4, 5, 10, 15, 20, 25, 30, 35, 40, 45, 50, or a number or a range between any two of these values, nucleotides in length.

### Molecular Labels

A barcode (e.g., a stochastic barcode) can comprise one or more molecular labels. Molecular labels can include barcode sequences. In some embodiments, a molecular label can comprise a nucleic acid sequence that provides identifying information for the specific type of target nucleic acid species hybridized to the barcode. A molecular label can comprise a nucleic acid sequence that provides a counter for the specific occurrence of the target nucleic acid species hybridized to the barcode (e.g., target-binding region).

In some embodiments, a diverse set of molecular labels are attached to a given solid support (e.g., a bead). In some embodiments, there can be, or be about, 10², 10³, 10⁴, 10⁵, 10⁶, 10⁷, 10⁸, 10⁹, or a number or a range between any two of these values, of unique molecular label sequences. For example, a plurality of barcodes can comprise about 6561 molecular labels with distinct sequences. As another example, a plurality of barcodes can comprise about 65536 molecular labels with distinct sequences. In some embodiments, there can be at least, or be at most, 10², 10³, 10⁴, 10⁵, 10⁶, 10⁷, 10⁸, or 10⁹, unique molecular label sequences. Barcodes with unique molecular label sequences can be attached to a given solid support (e.g., a bead).

For stochastic barcoding using a plurality of stochastic barcodes, the ratio of the number of different molecular label sequences and the number of occurrence of any of the targets can be, be about, at least, or is at most, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, 100:1, or a number or a range between any two of these values. A target can be an mRNA species comprising mRNA molecules with identical or nearly identical sequences.

A molecular label can be, be about, be at least, or be at most, 1, 2, 3, 4, 5, 10, 15, 20, 25, 30, 35, 40, 45, 50, or a number or a range between any two of these values, nucleotides in length.

### Target-Binding Region

A barcode can comprise one or more target binding regions, such as capture probes. In some embodiments, a target-binding region can hybridize with a target of interest. In some embodiments, the target binding regions can comprise a nucleic acid sequence that hybridizes specifically to a target (e.g., a target nucleic acid, target molecule, cellular nucleic acid to be analyzed), for example to a specific gene sequence. In some embodiments, a target binding region can comprise a nucleic acid sequence that can attach (e.g., hybridize) to a specific location of a specific target nucleic acid. In some embodiments, the target binding region can comprise a nucleic acid sequence that is capable of specific hybridization to a restriction enzyme site overhang (e.g., an EcoRI sticky-end overhang). The barcode can then ligate to any nucleic acid molecule comprising a sequence complementary to the restriction site overhang.

In some embodiments, a target binding region can comprise a non-specific target nucleic acid sequence. A non-specific target nucleic acid sequence can refer to a sequence that can bind to multiple target nucleic acids, independent of the specific sequence of the target nucleic acid. For example, target binding region can comprise a random multimer sequence, or an oligo(dT) sequence that hybridizes to the poly(A) tail on mRNA molecules. A random multimer sequence can be, for example, a random dimer, trimer, quatramer, pentamer, hexamer, septamer, octamer, nonamer, decamer, or higher multimer sequence of any length. In some embodiments, the target binding region is the same for all barcodes attached to a given bead. In some embodiments, the target binding regions for the plurality of barcodes attached to a given bead can comprise two or more different target binding sequences. A target binding region can be, be about, be at least about, or be at most about, 5, 10, 15, 20, 25, 30, 35, 40, 45, 50, or a number or a range between any two of these values, nucleotides in length.

In some embodiments, a target-binding region can comprise an oligo(dT) which can hybridize with mRNAs comprising polyadenylated ends. A target-binding region can be gene-specific. For example, a target-binding region can be configured to hybridize to a specific region of a target. A target-binding region can be, be about, be at least, or be at most, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these values, nucleotides in length. When a barcode comprises a gene-specific target-binding region, the barcode can be referred to herein as a gene-specific barcode.

### Universal Adaptor Primer

A barcode can comprise one or more universal adaptor primers. For example, a gene-specific barcode, such as a gene-specific stochastic barcode, can comprise a universal adaptor primer. A universal adaptor primer can refer to a nucleotide sequence that is universal across all barcodes. A universal adaptor primer can be used for building gene-specific barcodes. A universal adaptor primer can be, be about, at least, or be at most, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these nucleotides in length. A universal adaptor primer can be from 5-30 nucleotides in length.

### Linker

When a barcode comprises more than one of a type of label (e.g., more than one cell label or more than one barcode sequence, such as one molecular label), the labels may be interspersed with a linker label sequence. A linker label sequence can be, be about, be at least about, or be at most about, 5, 10, 11, 12, 13, 14, 15, 16, 17, 18, 19, 20, 25, 30, 35, 40, 45, 50 or more nucleotides in length. A linker label sequence can be used to facilitate the synthesis of the barcode. The linker label can comprise an error-correcting (e.g., Hamming) code.

### Solid Supports

Barcodes, such as stochastic barcodes, disclosed herein can, in some embodiments, be associated with a solid support. The solid support can be, for example, a synthetic particle. In some embodiments, some or all of the barcode sequences, such as molecular labels for stochastic barcodes (e.g., the first barcode sequences) of a plurality of barcodes (e.g., the first plurality of barcodes) on a solid support differ by at least one nucleotide. The cell labels of the barcodes on the same solid support can be the same. The cell labels of the barcodes on different solid supports can differ by at least one nucleotide. For example, first cell labels of a first plurality of barcodes on a first solid support can have the same sequence, and second cell labels of a second plurality of barcodes on a second solid support can have the same sequence. The first cell labels of the first plurality of barcodes on the first solid support and the second cell labels of the second plurality of barcodes on the second solid support can differ by at least one nucleotide. A cell label can be, for example, about 5-20 nucleotides long. A barcode sequence can be, for example, about 5-20 nucleotides long. The synthetic particle can be, for example, a bead.

The bead can be, for example, a silica gel bead, a controlled pore glass bead, a magnetic bead, a Dynabead, a Sephadex/Sepharose bead, a cellulose bead, a polystyrene bead, or any combination thereof. The bead can comprise a material such as polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, or any combination thereof. In some embodiments, the bead can be a polymeric bead, for example a deformable bead or a gel bead, functionalized with barcodes or stochastic barcodes (such as gel beads from 10X Genomics (San Francisco, CA). In some implementation, a gel bead can comprise a polymer based gels. Gel beads can be generated, for example, by encapsulating one or more polymeric precursors into droplets. Upon exposure of the polymeric precursors to an accelerator (e.g., tetramethylethylenediamine (TEMED)), a gel bead may be generated.

The particle can be degradable. For example, the polymeric bead can dissolve, melt, or degrade, for example, under a desired condition. The desired condition can include an environmental condition. The desired condition may result in the polymeric bead dissolving, melting, or degrading in a controlled manner. A gel bead may dissolve, melt, or degrade due to a chemical stimulus, a physical stimulus, a biological stimulus, a thermal stimulus, a magnetic stimulus, an electric stimulus, a light stimulus, or any combination thereof.

Analytes and/or reagents, such as oligonucleotide barcodes, for example, may be coupled/immobilized to the interior surface of a gel bead (e.g., the interior accessible via diffusion of an oligonucleotide barcode and/or materials used to generate an oligonucleotide barcode) and/or the outer surface of a gel bead or any other microcapsule described herein. Coupling/immobilization may be via any form of chemical bonding (e.g., covalent bond, ionic bond) or physical phenomena (e.g., Van der Waals forces, dipole-dipole interactions, etc.). In some embodiments, coupling/immobilization of a reagent to a gel bead or any other microcapsule described herein may be reversible, such as, for example, via a labile moiety (e.g., via a chemical cross-linker, including chemical cross-linkers described herein). Upon application of a stimulus, the labile moiety may be cleaved and the immobilized reagent set free. In some embodiments, the labile moiety is a disulfide bond. For example, in the case where an oligonucleotide barcode is immobilized to a gel bead via a disulfide bond, exposure of the disulfide bond to a reducing agent can cleave the disulfide bond and free the oligonucleotide barcode from the bead. The labile moiety may be included as part of a gel bead or microcapsule, as part of a chemical linker that links a reagent or analyte to a gel bead or microcapsule, and/or as part of a reagent or analyte. In some embodiments, at least one barcode of the plurality of barcodes can be immobilized on the particle, partially immobilized on the particle, enclosed in the particle, partially enclosed in the particle, or any combination thereof.

A gel bead can comprise a wide range of different polymers including but not limited to: polymers, heat sensitive polymers, photosensitive polymers, magnetic polymers, pH sensitive polymers, salt-sensitive polymers, chemically sensitive polymers, polyelectrolytes, polysaccharides, peptides, proteins, and/or plastics. Polymers may include but are not limited to materials such as poly(N-isopropylacrylamide) (PNIPAAm), poly(styrene sulfonate) (PSS), poly(allyl amine) (PAAm), poly(acrylic acid) (PAA), poly(ethylene imine) (PEI), poly(diallyldimethyl-ammonium chloride) (PDADMAC), poly(pyrolle) (PPy), poly(vinylpyrrolidone) (PVPON), poly(vinyl pyridine) (PVP), poly(methacrylic acid) (PMAA), poly(methyl methacrylate) (PMMA), polystyrene (PS), poly(tetrahydrofuran) (PTHF), poly(phthaladehyde) (PTHF), poly(hexyl viologen) (PHV), poly(L-lysine) (PLL), poly(L-arginine) (PARG), poly(lactic-co-glycolic acid) (PLGA).

Numerous chemical stimuli can be used to trigger the disruption, dissolution, or degradation of the beads. Examples of these chemical changes may include, but are not limited to pH-mediated changes to the bead wall, disintegration of the bead wall via chemical cleavage of crosslink bonds, triggered depolymerization of the bead wall, and bead wall switching reactions. Bulk changes may also be used to trigger disruption of the beads.

Bulk or physical changes to the microcapsule through various stimuli also offer many advantages in designing capsules to release reagents. Bulk or physical changes occur on a macroscopic scale, in which bead rupture is the result of mechano-physical forces induced by a stimulus. These processes may include, but are not limited to pressure induced rupture, bead wall melting, or changes in the porosity of the bead wall.

Biological stimuli may also be used to trigger disruption, dissolution, or degradation of beads. Generally, biological triggers resemble chemical triggers, but many examples use biomolecules, or molecules commonly found in living systems such as enzymes, peptides, saccharides, fatty acids, nucleic acids and the like. For example, beads may comprise polymers with peptide cross-links that are sensitive to cleavage by specific proteases. More specifically, one example may comprise a microcapsule comprising GFLGK peptide cross links. Upon addition of a biological trigger such as the protease Cathepsin B, the peptide cross links of the shell well are cleaved and the contents of the beads are released. In other cases, the proteases may be heat-activated. In another example, beads comprise a shell wall comprising cellulose. Addition of the hydrolytic enzyme chitosan serves as biologic trigger for cleavage of cellulosic bonds, depolymerization of the shell wall, and release of its inner contents.

The beads may be induced to release their contents upon the application of a thermal stimulus. A change in temperature can cause a variety changes to the beads. A change in heat may cause melting of a bead such that the bead wall disintegrates. In some cases, the heat may increase the internal pressure of the inner components of the bead such that the bead ruptures or explodes. In some cases, the heat may transform the bead into a shrunken dehydrated state. The heat may also act upon heat-sensitive polymers within the wall of a bead to cause disruption of the bead.

Inclusion of magnetic nanoparticles to the bead wall of microcapsules may allow triggered rupture of the beads as well as guide the beads in an array. A device of this disclosure can comprise magnetic beads for either purpose. For example, incorporation of Fe₃O₄ nanoparticles into polyelectrolyte containing beads triggers rupture in the presence of an oscillating magnetic field stimulus.

A bead may also be disrupted, dissolved, or degraded as the result of electrical stimulation. Similar to magnetic particles described in the previous section, electrically sensitive beads can allow for both triggered rupture of the beads as well as other functions such as alignment in an electric field, electrical conductivity or redox reactions. In one example, beads containing electrically sensitive material are aligned in an electric field such that release of inner reagents can be controlled. In other examples, electrical fields may induce redox reactions within the bead wall itself that may increase porosity.

A light stimulus may also be used to disrupt the beads. Numerous light triggers are possible and may include systems that use various molecules such as nanoparticles and chromophores capable of absorbing photons of specific ranges of wavelengths. For example, metal oxide coatings can be used as capsule triggers. UV irradiation of polyelectrolyte capsules coated with SiO₂ may result in disintegration of the bead wall. In yet another example, photo switchable materials such as azobenzene groups may be incorporated in the bead wall. Upon the application of UV or visible light, chemicals such as these undergo a reversible cis-to-trans isomerization upon absorption of photons. In this aspect, incorporation of photon switches can result in a bead wall that may disintegrate or become more porous upon the application of a light trigger.

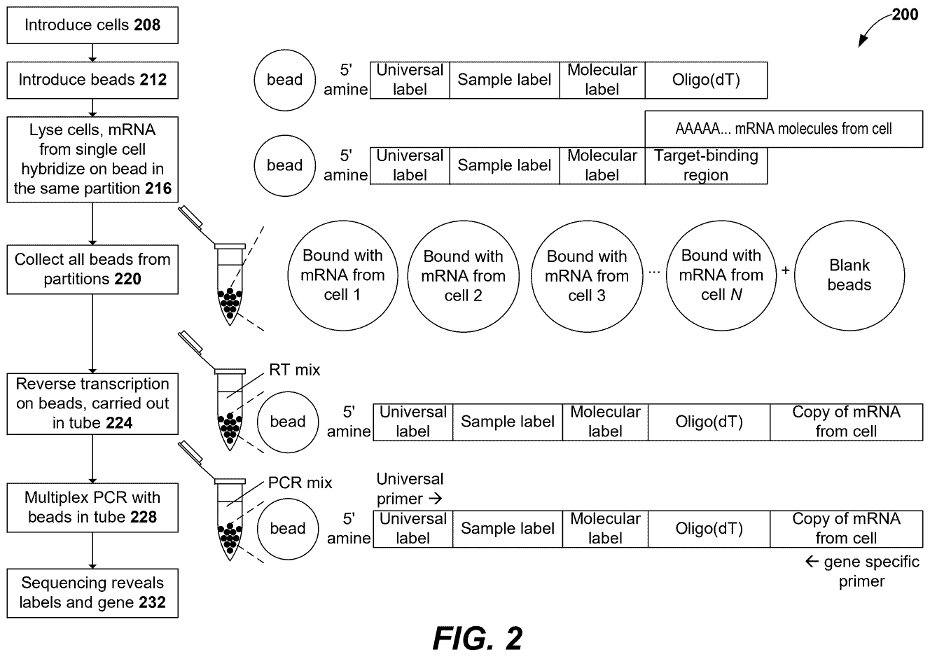

For example, in a non-limiting example of barcoding (e.g., stochastic barcoding) illustrated in FIG. 2, after introducing cells such as single cells onto a plurality of microwells of a microwell array at block 208, beads can be introduced onto the plurality of microwells of the microwell array at block 212. Each microwell can comprise one bead. The beads can comprise a plurality of barcodes. A barcode can comprise a 5′ amine region attached to a bead. The barcode can comprise a universal label, a barcode sequence (e.g., a molecular label), a target-binding region, or any combination thereof.

The barcodes disclosed herein can be associated with (e.g., attached to) a solid support (e.g., a bead). The barcodes associated with a solid support can each comprise a barcode sequence selected from at least 100 or 1000 barcode sequences with unique sequences. In some embodiments, different barcodes associated with a solid support can comprise barcode with different sequences. In some embodiments, a percentage of barcodes associated with a solid support comprises the same cell label. For example, the percentage can be, or be about 60%, 70%, 80%, 85%, 90%, 95%, 97%, 99%, 100%, or a number or a range between any two of these values. As another example, the percentage can be at least, or be at most 60%,70%, 80%, 85%, 90%, 95%,97%, 99%, or 100%. In some embodiments, barcodes associated with a solid support can have the same cell label. The barcodes associated with different solid supports can have different cell labels selected from at least 100 or 1000 cell labels with unique sequences.

The barcodes disclosed herein can be associated to (e.g., attached to) a solid support (e.g., a bead). In some embodiments, barcoding the plurality of targets in the sample can be performed with a solid support including a plurality of synthetic particles associated with the plurality of barcodes. In some embodiments, the solid support can include a plurality of synthetic particles associated with the plurality of barcodes. The spatial labels of the plurality of barcodes on different solid supports can differ by at least one nucleotide. The solid support can, for example, include the plurality of barcodes in two dimensions or three dimensions. The synthetic particles can be beads. The beads can be silica gel beads, controlled pore glass beads, magnetic beads, Dynabeads, Sephadex/Sepharose beads, cellulose beads, polystyrene beads, or any combination thereof. The solid support can include a polymer, a matrix, a hydrogel, a needle array device, an antibody, or any combination thereof. In some embodiments, the solid supports can be free floating. In some embodiments, the solid supports can be embedded in a semi-solid or solid array. The barcodes may not be associated with solid supports. The barcodes can be individual nucleotides. The barcodes can be associated with a substrate.

As used herein, the terms “tethered,” “attached,” and “immobilized,” are used interchangeably, and can refer to covalent or non-covalent means for attaching barcodes to a solid support. Any of a variety of different solid supports can be used as solid supports for attaching pre-synthesized barcodes or for in situ solid-phase synthesis of barcode.

The solid support can be a bead. The bead can comprise one or more types of solid, porous, or hollow sphere, ball, bearing, cylinder, or other similar configuration which a nucleic acid can be immobilized (e.g., covalently or non-covalently). The bead can be, for example, composed of plastic, ceramic, metal, polymeric material, or any combination thereof. A bead can be, or comprise, a discrete particle that is spherical (e.g., microspheres) or have a non-spherical or irregular shape, such as cubic, cuboid, pyramidal, cylindrical, conical, oblong, or disc-shaped, and the like. In some embodiments, a bead can be non-spherical in shape.

Beads can comprise a variety of materials including, but not limited to, paramagnetic materials (e.g., magnesium, molybdenum, lithium, and tantalum), superparamagnetic materials (e.g., ferrite (Fe₃O₄; magnetite) nanoparticles), ferromagnetic materials (e.g., iron, nickel, cobalt, some alloys thereof, and some rare earth metal compounds), ceramic, plastic, glass, polystyrene, silica, methylstyrene, acrylic polymers, titanium, latex, Sepharose, agarose, hydrogel, polymer, cellulose, nylon, or any combination thereof. In some embodiments, the bead (e.g., the bead to which the labels are attached) is a hydrogel bead. In some embodiments, the bead comprises hydrogel.

Some embodiments disclosed herein include one or more particles (e.g., beads). Each of the particles can comprise a plurality of oligonucleotides (e.g., barcodes). Each of the plurality of oligonucleotides can comprise a barcode sequence (e.g., a molecular label sequence), a cell label, and a target-binding region (e.g., an oligo(dT) sequence, a gene-specific sequence, a random multimer, or a combination thereof). The cell label sequence of each of the plurality of oligonucleotides can be the same. The cell label sequences of oligonucleotides on different particles can be different such that the oligonucleotides on different particles can be identified. The number of different cell label sequences can be different in different implementations. In some embodiments, the number of cell label sequences can be, be about, be at least, or be at most, 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 106, 107, 108, 109, a number or a range between any two of these values, or more. In some embodiments, no more than 1, 2, 3, 4, 5, 6, 7, 8, 9, 10, 20, 30, 40, 50, 60, 70, 80, 90, 100, 200, 300, 400, 500, 600, 700, 800, 900, 1000, or more of the plurality of the particles include oligonucleotides with the same cell sequence. In some embodiment, the plurality of particles that include oligonucleotides with the same cell sequence can be at most 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or more. In some embodiments, none of the plurality of the particles has the same cell label sequence.

The plurality of oligonucleotides on each particle can comprise different barcode sequences (e.g., molecular labels). In some embodiments, the number of barcode sequences can be, be about, be at least, or be at most 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 106, 107, 108, 109, or a number or a range between any two of these values. For example, at least 100 of the plurality of oligonucleotides comprise different barcode sequences. As another example, in a single particle, at least 100, 500, 1000, 5000, 10000, 15000, 20000, 50000, a number or a range between any two of these values, or more of the plurality of oligonucleotides comprise different barcode sequences. Some embodiments provide a plurality of the particles comprising barcodes. In some embodiments, the ratio of an occurrence (or a copy or a number) of a target to be labeled and the different barcode sequences can be at least 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, or more. In some embodiments, each of the plurality of oligonucleotides further comprises a sample label, a universal label, or both. The particle can be, for example, a nanoparticle or microparticle.

The size of the beads can vary. For example, the diameter of the bead can range from 0.1 micrometer to50 micrometers. In some embodiments, the diameter of the bead can be, or be about, 0.1, 0.5, 1, 2, 3, 4, 5, 6, 7, 8, 9, 10, 20, 30, 40, or 50 micrometers, or a number or a range between any two of these values.

The diameter of the bead can be related to the diameter of the wells of the substrate. In some embodiments, the diameter of the bead can be, or be about, 10%, 20%, 30%, 40%, 50%, 60%, 70%, 80%, 90%, 100%, or a number or a range between any two of these values, longer or shorter than the diameter of the well. The diameter of the beads can be related to the diameter of a cell (e.g., a single cell entrapped by a well of the substrate). In some embodiments, the diameter of the bead can be at least, or be at most, 10%, 20%, 30%, 40%, 50%, 60%, 70%, 80%, 90%, or 100% longer or shorter than the diameter of the well. The diameter of the beads can be related to the diameter of a cell (e.g., a single cell entrapped by a well of the substrate). In some embodiments, the diameter of the bead can be, be about, be at least, or be at most, 10%, 20%, 30%, 40%,50%, 60%, 70%, 80%, 90%, 100%, 150%, 200%, 250%, 300%, or a number or a range between any two of these values, longer or shorter than the diameter of the cell.

A bead can be attached to and/or embedded in a substrate. A bead can be attached to and/or embedded in a gel, hydrogel, polymer and/or matrix. The spatial position of a bead within a substrate (e.g., gel, matrix, scaffold, or polymer) can be identified using the spatial label present on the barcode on the bead which can serve as a location address.

Examples of beads can include, but are not limited to, streptavidin beads, agarose beads, magnetic beads, Dynabeads®, MACS® microbeads, antibody conjugated beads (e.g., anti-immunoglobulin microbeads), protein A conjugated beads, protein G conjugated beads, protein A/G conjugated beads, protein L conjugated beads, oligo(dT) conjugated beads, silica beads, silica-like beads, anti-biotin microbeads, anti-fluorochrome microbeads, and BcMag™ Carboxyl-Terminated Magnetic Beads.

A bead can be associated with (e.g., impregnated with) quantum dots or fluorescent dyes to make it fluorescent in one fluorescence optical channel or multiple optical channels. A bead can be associated with iron oxide or chromium oxide to make it paramagnetic or ferromagnetic. Beads can be identifiable. For example, a bead can be imaged using a camera. A bead can have a detectable code associated with the bead. For example, a bead can comprise a barcode. A bead can change size, for example, due to swelling in an organic or inorganic solution. A bead can be hydrophobic. A bead can be hydrophilic. A bead can be biocompatible.

A solid support (e.g., a bead) can be visualized. The solid support can comprise a visualizing tag (e.g., fluorescent dye). A solid support (e.g., a bead) can be etched with an identifier (e.g., a number). The identifier can be visualized through imaging the beads.

A solid support can comprise an insoluble, semi-soluble, or insoluble material. A solid support can be referred to as “functionalized” when it includes a linker, a scaffold, a building block, or other reactive moiety attached thereto, whereas a solid support may be “nonfunctionalized” when it lacks such a reactive moiety attached thereto. The solid support can be employed free in solution, such as in a microtiter well format; in a flow-through format, such as in a column; or in a dipstick.

The solid support can comprise a membrane, paper, plastic, coated surface, flat surface, glass, slide, chip, or any combination thereof. A solid support can take the form of resins, gels, microspheres, or other geometric configurations. A solid support can comprise silica chips, microparticles, nanoparticles, plates, arrays, capillaries, flat supports such as glass fiber filters, glass surfaces, metal surfaces (steel, gold silver, aluminum, silicon and copper), glass supports, plastic supports, silicon supports, chips, filters, membranes, microwell plates, slides, plastic materials including multiwell plates or membranes (e.g., formed of polyethylene, polypropylene, polyamide, polyvinylidenedifluoride), and/or wafers, combs, pins or needles (e.g., arrays of pins suitable for combinatorial synthesis or analysis) or beads in an array of pits or nanoliter wells of flat surfaces such as wafers (e.g., silicon wafers), wafers with pits with or without filter bottoms.

The solid support can comprise a polymer matrix (e.g., gel, hydrogel). The polymer matrix may be able to permeate intracellular space (e.g., around organelles). The polymer matrix may able to be pumped throughout the circulatory system.

As used herein, a substrate can refer to a type of solid support. A substrate can refer to a solid support that can comprise barcodes or stochastic barcodes of the disclosure. A substrate can, for example, comprise a plurality of microwells. For example, a substrate can be a well array comprising two or more microwells. In some embodiments, a microwell can comprise a small reaction chamber of defined volume. In some embodiments, a microwell can entrap one or more cells. In some embodiments, a microwell can entrap only one cell. In some embodiments, a microwell can entrap one or more solid supports. In some embodiments, a microwell can entrap only one solid support. In some embodiments, a microwell entraps a single cell and a single solid support (e.g., a bead). A microwell can comprise barcode reagents of the disclosure.

### Methods of Barcoding

The disclosure provides for methods for estimating the number of distinct targets at distinct locations in a physical sample (e.g., tissue, organ, tumor, cell). The methods can comprise placing barcodes (e.g., stochastic barcodes) in close proximity with the sample, lysing the sample, associating distinct targets with the barcodes, amplifying the targets and/or digitally counting the targets. The method can further comprise analyzing and/or visualizing the information obtained from the spatial labels on the barcodes. In some embodiments, a method comprises visualizing the plurality of targets in the sample. Mapping the plurality of targets onto the map of the sample can include generating a two dimensional map or a three dimensional map of the sample. The two dimensional map and the three dimensional map can be generated prior to or after barcoding (e.g., stochastically barcoding) the plurality of targets in the sample. Visualizing the plurality of targets in the sample can include mapping the plurality of targets onto a map of the sample. Mapping the plurality of targets onto the map of the sample can include generating a two dimensional map or a three dimensional map of the sample. The two dimensional map and the three dimensional map can be generated prior to or after barcoding the plurality of targets in the sample. in some embodiments, the two dimensional map and the three dimensional map can be generated before or after lysing the sample. Lysing the sample before or after generating the two dimensional map or the three dimensional map can include heating the sample, contacting the sample with a detergent, changing the pH of the sample, or any combination thereof.

Barcoding the plurality of targets can comprise hybridizing a plurality of barcodes with a plurality of targets to create barcoded targets (e.g., stochastically barcoded targets). Barcoding the plurality of targets can comprise generating an indexed library of the barcoded targets. Generating an indexed library of the barcoded targets can be performed with a solid support comprising the plurality of barcodes (e.g., stochastic barcodes).

### Contacting a Sample and a Barcode

The disclosure provides for methods for contacting a sample (e.g., cells) to a substrate of the disclosure. A sample comprising, for example, a cell, organ, or tissue thin section, can be contacted to barcodes (e.g., stochastic barcodes). The cells can be contacted, for example, by gravity flow wherein the cells can settle and create a monolayer. The sample can be a tissue thin section. The thin section can be placed on the substrate. The sample can be one-dimensional (e.g., forms a planar surface). The sample (e.g., cells) can be spread across the substrate, for example, by growing/culturing the cells on the substrate.

When barcodes are in close proximity to targets, the targets can hybridize to the barcode. The barcodes can be contacted at a non-depletable ratio such that each distinct target can associate with a distinct barcode of the disclosure. To ensure efficient association between the target and the barcode, the targets can be cross-linked to barcode.

### Cell Lysis

Following the distribution of cells and barcodes, the cells can be lysed to liberate the target molecules. Cell lysis can be accomplished by any of a variety of means, for example, by chemical or biochemical means, by osmotic shock, or by means of thermal lysis, mechanical lysis, or optical lysis. Cells can be lysed by addition of a cell lysis buffer comprising a detergent (e.g., SDS, Li dodecyl sulfate, Triton X-100, Tween-20, or NP-40), an organic solvent (e.g., methanol or acetone), or digestive enzymes (e.g., proteinase K, pepsin, or trypsin), or any combination thereof. To increase the association of a target and a barcode, the rate of the diffusion of the target molecules can be altered by for example, reducing the temperature and/or increasing the viscosity of the lysate.

In some embodiments, the sample can be lysed using a filter paper. The filter paper can be soaked with a lysis buffer on top of the filter paper. The filter paper can be applied to the sample with pressure which can facilitate lysis of the sample and hybridization of the targets of the sample to the substrate.

In some embodiments, lysis can be performed by mechanical lysis, heat lysis, optical lysis, and/or chemical lysis. Chemical lysis can include the use of digestive enzymes such as proteinase K, pepsin, and trypsin. Lysis can be performed by the addition of a lysis buffer to the substrate. A lysis buffer can comprise Tris HCl. A lysis buffer can comprise at least about, or at most about, 0.01, 0.05, 0.1, 0.5, or 1 M or more Tris HCl. A lysis buffer can comprise about 0.1 M Tris HCl. The pH of the lysis buffer can be, be about, be at least about, or be at most about 1, 2, 3, 4, 5, 5.5, 6, 6.5, 7, 7.5, 8, 8.5, 9, 9.5, 10, or a number or a range between any two of these values. The lysis buffer can comprise a salt (e.g., LiCl). The concentration of salt in the lysis buffer can be at least about 0.1, 0.5, or 1 M or more. The concentration of salt in the lysis buffer can be at most about 0.1, 0.5, or 1 M or more. In some embodiments, the concentration of salt in the lysis buffer is about 0.5M. The lysis buffer can comprise a detergent (e.g., SDS, Li dodecyl sulfate, triton X, tween, NP-40). The concentration of the detergent in the lysis buffer can be at least about, or at most about, 0.0001%, 0.0005%, 0.001%, 0.005%, 0.01%, 0.05%, 0.1%, 0.5%, 1%, 2%, 3%, 4%, 5%, 6%, or 7%, or more. In some embodiments, the concentration of the detergent in the lysis buffer is about 1% Li dodecyl sulfate. The time used in the method for lysis can be dependent on the amount of detergent used. In some embodiments, the more detergent used, the less time needed for lysis. The lysis buffer can comprise a chelating agent (e.g., EDTA, EGTA). The concentration of a chelating agent in the lysis buffer can be at least about 1, 5, 10, 15, 20, 25, or 30 mM or more. The concentration of a chelating agent in the lysis buffer can be at most about 1, 5, 10, 15, 20, 25, or 30 mM or more. In some embodiments, the concentration of chelating agent in the lysis buffer is about 10 mM. The lysis buffer can comprise a reducing reagent (e.g., beta-mercaptoethanol, DTT). The concentration of the reducing reagent in the lysis buffer can be at least about, at most about, 1, 5, 10, 15, or 20 mM or more. In some embodiments, a lysis buffer can comprise about 0.1M TrisHCl, about pH 7.5, about 0.5M LiCl, about 1% lithium dodecyl sulfate, about 10 mM EDTA, and about 5 mM DTT.

Lysis can be performed at a temperature of about 4, 10, 15, 20, 25, or 30° C. Lysis can be performed for about 1, 5, 10, 15, or 20 or more minutes. A lysed cell can comprise at least about, or at most about, 100000, 200000, 300000, 400000, 500000, 600000, or 700000 or more target nucleic acid molecules.

### Attachment of Barcodes to Target Nucleic Acid Molecules

Following lysis of the cells and release of nucleic acid molecules therefrom, the nucleic acid molecules can randomly associate with the barcodes of the co-localized solid support. Association can comprise hybridization of a barcode's target recognition region to a complementary portion of the target nucleic acid molecule (e.g., oligo(dT) of the barcode can interact with a poly(A) tail of a target). The assay conditions used for hybridization (e.g., buffer pH, ionic strength, temperature) can be chosen to promote formation of specific, stable hybrids. In some embodiments, the nucleic acid molecules released from the lysed cells can associate with the plurality of probes on the substrate (e.g., hybridize with the probes on the substrate). When the probes comprise oligo(dT), mRNA molecules can hybridize to the probes and be reverse transcribed. The oligo(dT) portion of the oligonucleotide can act as a primer for first strand synthesis of the cDNA molecule. For example, in a non-limiting example of barcoding illustrated in FIG. 2, at block 216, mRNA molecules can hybridize to barcodes on beads. For example, single-stranded nucleotide fragments can hybridize to the target-binding regions of barcodes.

Attachment can further comprise ligation of a barcode's target recognition region and a portion of the target nucleic acid molecule. For example, the target binding region can comprise a nucleic acid sequence that can be capable of specific hybridization to a restriction site overhang (e.g., an EcoRI sticky-end overhang). The assay procedure can further comprise treating the target nucleic acids with a restriction enzyme (e.g., EcoRI) to create a restriction site overhang. The barcode can then be ligated to any nucleic acid molecule comprising a sequence complementary to the restriction site overhang. A ligase (e.g., T4 DNA ligase) can be used to join the two fragments.

For example, in a non-limiting example of barcoding illustrated in FIG. 2, at block 220, the labeled targets from a plurality of cells (or a plurality of samples) (e.g., target-barcode molecules) can be subsequently pooled, for example, into a tube. The labeled targets can be pooled by, for example, retrieving the barcodes and/or the beads to which the target-barcode molecules are attached.

The retrieval of solid support-based collections of attached target-barcode molecules can be implemented by use of magnetic beads and an externally-applied magnetic field. Once the target-barcode molecules have been pooled, all further processing can proceed in a single reaction vessel. Further processing can include, for example, reverse transcription reactions, amplification reactions, cleavage reactions, dissociation reactions, and/or nucleic acid extension reactions. Further processing reactions can be performed within the microwells, that is, without first pooling the labeled target nucleic acid molecules from a plurality of cells.

Reverse Transcription The disclosure provides for a method to create a target-barcode conjugate using reverse transcription (e.g., at block 224 of FIG. 2). The target-barcode conjugate can comprise the barcode and a complementary sequence of all or a portion of the target nucleic acid (i.e., a barcoded cDNA molecule, such as a stochastically barcoded cDNA molecule). Reverse transcription of the associated RNA molecule can occur by the addition of a reverse transcription primer along with the reverse transcriptase. The reverse transcription primer can be an oligo(dT) primer, a random hexanucleotide primer, or a target-specific oligonucleotide primer. Oligo(dT) primers can be, or can be about, 12-18 nucleotides in length and bind to the endogenous poly(A) tail at the 3′ end of mammalian mRNA. Random hexanucleotide primers can bind to mRNA at a variety of complementary sites. Target-specific oligonucleotide primers typically selectively prime the mRNA of interest.

In some embodiments, reverse transcription of the labeled-RNA molecule can occur by the addition of a reverse transcription primer. In some embodiments, the reverse transcription primer is an oligo(dT) primer, random hexanucleotide primer, or a target-specific oligonucleotide primer. Generally, oligo(dT) primers are 12-18 nucleotides in length and bind to the endogenous poly(A) tail at the 3′ end of mammalian mRNA. Random hexanucleotide primers can bind to mRNA at a variety of complementary sites. Target-specific oligonucleotide primers typically selectively prime the mRNA of interest.

Reverse transcription can occur repeatedly to produce multiple labeled-cDNA molecules. The methods disclosed herein can comprise conducting at least about, or at most about, 1, 2, 3, 4, 5, 6, 7, 8, 9, 10, 11, 12, 13, 14, 15, 16, 17, 18, 19, or 20 reverse transcription reactions.

### Amplification

One or more nucleic acid amplification reactions (e.g., at block 228 of FIG. 2) can be performed to create multiple copies of the labeled target nucleic acid molecules. Amplification can be performed in a multiplexed manner, wherein multiple target nucleic acid sequences are amplified simultaneously. The amplification reaction can be used to add sequencing adaptors to the nucleic acid molecules. The amplification reactions can comprise amplifying at least a portion of a sample label, if present. The amplification reactions can comprise amplifying at least a portion of the cellular label and/or barcode sequence (e.g., a molecular label). The amplification reactions can comprise amplifying at least a portion of a sample tag, a cell label, a spatial label, a barcode sequence (e.g., a molecular label), a target nucleic acid, or a combination thereof. The amplification reactions can comprise amplifying 0.5%, 1%, 2%, 3%, 4%, 5%, 6%, 7%, 8%, 9%, 10%, 5%, 20%, 25%, 30%, 35%, 40%, 45%, 05%,55%, 60%, 65%, 70%, 75%, 80%, 85%, 90%, 95%, 97%, 100%, or a range or a number between any two of these values, of the plurality of nucleic acids. The method can further comprise conducting one or more cDNA synthesis reactions to produce one or more cDNA copies of target-barcode molecules comprising a sample label, a cell label, a spatial label, and/or a barcode sequence (e.g., a molecular label).

In some embodiments, amplification can be performed using a polymerase chain reaction (PCR). As used herein, PCR can refer to a reaction for the in vitro amplification of specific DNA sequences by the simultaneous primer extension of complementary strands of DNA. As used herein, PCR can encompass derivative forms of the reaction, including but not limited to, RT-PCR, real-time PCR, nested PCR, quantitative PCR, multiplexed PCR, digital PCR, and assembly PCR.

Amplification of the labeled nucleic acids can comprise non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), and a QB replicase (QB) method, use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and ramification extension amplification (RAM). In some embodiments, the amplification does not produce circularized transcripts.

In some embodiments, the methods disclosed herein further comprise conducting a polymerase chain reaction on the labeled nucleic acid (e.g., labeled-RNA, labeled-DNA, labeled-cDNA) to produce a labeled amplicon (e.g., a stochastically labeled amplicon). The labeled amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample label, a spatial label, a cell label, and/or a barcode sequence (e.g., a molecular label). The labeled amplicon can be a single-stranded molecule. The single-stranded molecule can comprise DNA, RNA, or a combination thereof. The nucleic acids of the disclosure can comprise synthetic or altered nucleic acids.

Amplification can comprise use of one or more non-natural nucleotides. Non-natural nucleotides can comprise photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise, for example, 1, 2, 3, 4, 5, 6, 7, 8, 9, 10, 11, 12, 13, 14, or 15 or more nucleotides. The one or more primers can comprise at least 1, 2, 3, 4, 5, 6, 7, 8, 9, 10, 11, 12, 13, 14, or 15 or more nucleotides. The one or more primers can anneal to at least a portion of the plurality of labeled targets (e.g., stochastically labeled targets). The one or more primers can anneal to the 3′ end or 5′ end of the plurality of labeled targets. The one or more primers can anneal to an internal region of the plurality of labeled targets. The internal region can be at least about, or at most about, 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled targets. The one or more primers can comprise a fixed panel of primers. The one or more primers can comprise at least one or more custom primers. The one or more primers can comprise at least one or more control primers. The one or more primers can comprise at least one or more gene-specific primers.

The one or more primers can comprise a universal primer. The universal primer can anneal to a universal primer binding site. The one or more custom primers can anneal to a first sample label, a second sample label, a spatial label, a cell label, a barcode sequence (e.g., a molecular label), a target, or any combination thereof. The one or more primers can comprise a universal primer and a custom primer. The custom primer can be designed to amplify one or more targets. The targets can comprise a subset of the total nucleic acids in one or more samples. The targets can comprise a subset of the total labeled targets in one or more samples. The one or more primers can comprise at least 96 or more custom primers, at least 960 or more custom primers, or at least 9600 or more custom primers. The one or more custom primers can anneal to two or more different labeled nucleic acids. The two or more different labeled nucleic acids can correspond to one or more genes.

Any amplification scheme can be used in the methods of the present disclosure. For example, in one scheme, the first round PCR can amplify molecules attached to the bead using a gene specific primer and a primer against the universal Illumina sequencing primer 1 sequence. The second round of PCR can amplify the first PCR products using a nested gene specific primer flanked by Illumina sequencing primer 2 sequence, and a primer against the universal Illumina sequencing primer 1 sequence. The third round of PCR adds P5 and P7 and sample index to turn PCR products into an Illumina sequencing library. Sequencing using 150 pb×2 sequencing can reveal the cell label and barcode sequence (e.g., molecular label) on read 1, the gene on read 2, and the sample index on index 1 read.

In some embodiments, nucleic acids can be removed from the substrate using chemical cleavage. For example, a chemical group or a modified base present in a nucleic acid can be used to facilitate its removal from a solid support. For example, an enzyme can be used to remove a nucleic acid from a substrate. For example, a nucleic acid can be removed from a substrate through a restriction endonuclease digestion. For example, treatment of a nucleic acid containing a dUTP or ddUTP with uracil-d-glycosylase (UDG) can be used to remove a nucleic acid from a substrate. For example, a nucleic acid can be removed from a substrate using an enzyme that performs nucleotide excision, such as a base excision repair enzyme, such as an apurinic/apyrimidinic (AP) endonuclease. In some embodiments, a nucleic acid can be removed from a substrate using a photocleavable group and light. In some embodiments, a cleavable linker can be used to remove a nucleic acid from the substrate. For example, the cleavable linker can comprise at least one of biotin/avidin, biotin/streptavidin, biotin/neutravidin, Ig-protein A, a photo-labile linker, acid or base labile linker group, or an aptamer.

When the probes are gene-specific, the molecules can hybridize to the probes and be reverse transcribed and/or amplified. In some embodiments, after the nucleic acid has been synthesized (e.g., reverse transcribed), it can be amplified. Amplification can be performed in a multiplex manner, wherein multiple target nucleic acid sequences are amplified simultaneously. Amplification can add sequencing adaptors to the nucleic acid.

In some embodiments, amplification can be performed on the substrate, for example, with bridge amplification. cDNAs can be homopolymer tailed in order to generate a compatible end for bridge amplification using oligo(dT) probes on the substrate. In bridge amplification, the primer that is complementary to the 3′ end of the template nucleic acid can be the first primer of each pair that is covalently attached to the solid particle. When a sample containing the template nucleic acid is contacted with the particle and a single thermal cycle is performed, the template molecule can be annealed to the first primer and the first primer is elongated in the forward direction by addition of nucleotides to form a duplex molecule consisting of the template molecule and a newly formed DNA strand that is complementary to the template. In the heating step of the next cycle, the duplex molecule can be denatured, releasing the template molecule from the particle and leaving the complementary DNA strand attached to the particle through the first primer. In the annealing stage of the annealing and elongation step that follows, the complementary strand can hybridize to the second primer, which is complementary to a segment of the complementary strand at a location removed from the first primer. This hybridization can cause the complementary strand to form a bridge between the first and second primers secured to the first primer by a covalent bond and to the second primer by hybridization. In the elongation stage, the second primer can be elongated in the reverse direction by the addition of nucleotides in the same reaction mixture, thereby converting the bridge to a double-stranded bridge. The next cycle then begins, and the double-stranded bridge can be denatured to yield two single-stranded nucleic acid molecules, each having one end attached to the particle surface via the first and second primers, respectively, with the other end of each unattached. In the annealing and elongation step of this second cycle, each strand can hybridize to a further complementary primer, previously unused, on the same particle, to form new single-strand bridges. The two previously unused primers that are now hybridized elongate to convert the two new bridges to double-strand bridges.

The amplification reactions can comprise amplifying at least 1%, 2%, 3%, 4%, 5%, 6%, 7%, 8%, 9%, 0%, 15%, 20%, 25%, 30%, 35%, 40%, 45%, 50%, 55%, 60%, 65%, 70%, 75%, 80%, 85%, 90%,95%, 97%, or 100% of the plurality of nucleic acids.

Amplification of the labeled nucleic acids can comprise PCR-based methods or non-PCR based methods. Amplification of the labeled nucleic acids can comprise exponential amplification of the labeled nucleic acids. Amplification of the labeled nucleic acids can comprise linear amplification of the labeled nucleic acids. Amplification can be performed by polymerase chain reaction (PCR). PCR can refer to a reaction for the in vitro amplification of specific DNA sequences by the simultaneous primer extension of complementary strands of DNA. PCR can encompass derivative forms of the reaction, including but not limited to, RT-PCR, real-time PCR, nested PCR, quantitative PCR, multiplexed PCR, digital PCR, suppression PCR, semi-suppressive PCR and assembly PCR.

Amplification of the labeled nucleic acids can comprise non-PCR based methods, including but not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), a QB replicase (QB), use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and/or ramification extension amplification (RAM).

In some embodiments, the methods disclosed herein further comprise conducting a nested polymerase chain reaction on the amplified amplicon (e.g., target). The amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample tag or molecular identifier label. Alternatively, the amplicon can be a single-stranded molecule. The single-stranded molecule can comprise DNA, RNA, or a combination thereof. The nucleic acids of the present invention can comprise synthetic or altered nucleic acids.

In some embodiments, the method comprises repeatedly amplifying the labeled nucleic acid to produce multiple amplicons. The methods disclosed herein can comprise conducting at least about, at most about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 amplification reactions.

Amplification can further comprise adding one or more control nucleic acids to one or more samples comprising a plurality of nucleic acids. Amplification can further comprise adding one or more control nucleic acids to a plurality of nucleic acids. The control nucleic acids can comprise a control label.

Amplification can comprise use of one or more non-natural nucleotides. Non-natural nucleotides can comprise photolabile and/or triggerable nucleotides. Examples of non-natural nucleotides include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise one or more oligonucleotides. The one or more oligonucleotides can comprise at least about 7-9 nucleotides. The one or more oligonucleotides can comprise less than 12-15 nucleotides. The one or more primers can anneal to at least a portion of the plurality of labeled nucleic acids. The one or more primers can anneal to the 3′ end and/or 5′ end of the plurality of labeled nucleic acids. The one or more primers can anneal to an internal region of the plurality of labeled nucleic acids. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled nucleic acids. The one or more primers can comprise a fixed panel of primers. The one or more primers can comprise at least one or more custom primers. The one or more primers can comprise at least one or more control primers. The one or more primers can comprise at least one or more housekeeping gene primers. The one or more primers can comprise a universal primer. The universal primer can anneal to a universal primer binding site. The one or more custom primers can anneal to the first sample tag, the second sample tag, the molecular identifier label, the nucleic acid or a product thereof. The one or more primers can comprise a universal primer and a custom primer. The custom primer can be designed to amplify one or more target nucleic acids. The target nucleic acids can comprise a subset of the total nucleic acids in one or more samples. In some embodiments, the primers are the probes attached to the array of the disclosure.

In some embodiments, barcoding (e.g., stochastically barcoding) the plurality of targets in the sample further comprises generating an indexed library of the barcoded targets (e.g., stochastically barcoded targets) or barcoded fragments of the targets. The barcode sequences of different barcodes (e.g., the molecular labels of different stochastic barcodes) can be different from one another. Generating an indexed library of the barcoded targets includes generating a plurality of indexed polynucleotides from the plurality of targets in the sample. For example, for an indexed library of the barcoded targets comprising a first indexed target and a second indexed target, the label region of the first indexed polynucleotide can differ from the label region of the second indexed polynucleotide by, by about, by at least, or by at most, 1, 2, 3, 4, 5, 6, 7, 8, 9, 10, 20, 30, 40, 50, or a number or a range between any two of these values, nucleotides. In some embodiments, generating an indexed library of the barcoded targets includes contacting a plurality of targets, for example mRNA molecules, with a plurality of oligonucleotides including a poly(T) region and a label region; and conducting a first strand synthesis using a reverse transcriptase to produce single-strand labeled cDNA molecules each comprising a cDNA region and a label region, wherein the plurality of targets includes at least two mRNA molecules of different sequences and the plurality of oligonucleotides includes at least two oligonucleotides of different sequences. Generating an indexed library of the barcoded targets can further comprise amplifying the single-strand labeled cDNA molecules to produce double-strand labeled cDNA molecules; and conducting nested PCR on the double-strand labeled cDNA molecules to produce labeled amplicons. In some embodiments, the method can include generating an adaptor-labeled amplicon.

Barcoding (e.g., stochastic barcoding) can include using nucleic acid barcodes or tags to label individual nucleic acid (e.g., DNA or RNA) molecules. In some embodiments, it includes adding DNA barcodes or tags to cDNA molecules as they are generated from mRNA. Nested PCR can be performed to minimize PCR amplification bias. Adaptors can be added for sequencing using, for example, next generation sequencing (NGS). The sequencing results can be used to determine cell labels, molecular labels, and sequences of nucleotide fragments of the one or more copies of the targets, for example at block 232 of FIG. 2.

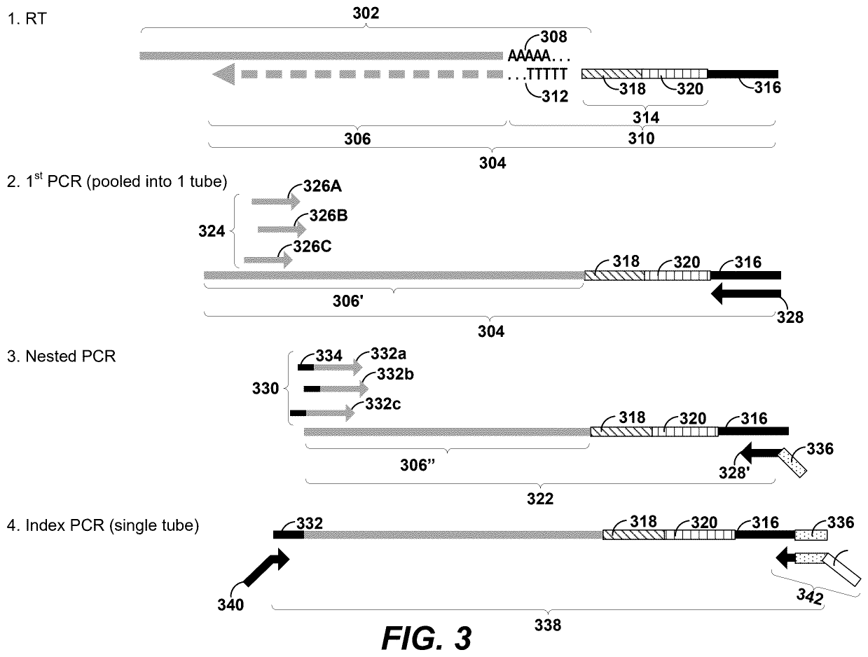

FIG. 3 is a schematic illustration showing a non-limiting exemplary process of generating an indexed library of the barcoded targets (e.g., stochastically barcoded targets), such as barcoded mRNAs or fragments thereof. As shown in step 1, the reverse transcription process can encode each mRNA molecule with a unique molecular label, a cell label, and a universal PCR site. In particular, RNA molecules 302 can be reverse transcribed to produce labeled cDNA molecules 304, including a cDNA region 306, by hybridization (e.g., stochastic hybridization) of a set of barcodes (e.g., stochastic barcodes) 310 to the poly(A) tail region 308 of the RNA molecules 302. Each of the barcodes 310 can comprise a target-binding region, for example a poly(dT) region 312, a label region 314 (e.g., a barcode sequence or a molecule), and a universal PCR region 316.

In some embodiments, the cell label can include 3 to 20 nucleotides. In some embodiments, the molecular label can include 3 to 20 nucleotides. In some embodiments, each of the plurality of stochastic barcodes further comprises one or more of a universal label and a cell label, wherein universal labels are the same for the plurality of stochastic barcodes on the solid support and cell labels are the same for the plurality of stochastic barcodes on the solid support. In some embodiments, the universal label can include 3 to 20 nucleotides. In some embodiments, the cell label comprises 3 to 20 nucleotides.

In some embodiments, the label region 314 can include a barcode sequence or a molecular label 318 and a cell label 320. In some embodiments, the label region 314 can include one or more of a universal label, a dimension label, and a cell label. The barcode sequence or molecular label 318 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The cell label 320 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The universal label can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. Universal labels can be the same for the plurality of stochastic barcodes on the solid support and cell labels are the same for the plurality of stochastic barcodes on the solid support. The dimension label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length.

In some embodiments, the label region 314 can comprise, comprise about, comprise at least, or comprise at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different labels, such as a barcode sequence or a molecular label 318 and a cell label 320. Each label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length. A set of barcodes or stochastic barcodes 310 can contain, contain about, contain at least, or can be at most, 10, 20, 40, 50, 70, 80, 90, 102, 103, 104, 105, 106, 107, 108, 109, 1010, 1011, 1012, 1013, 1014, 1015, 1020, or a number or a range between any of these values, barcodes or stochastic barcodes 310. And the set of barcodes or stochastic barcodes 310 can, for example, each contain a unique label region 314. The labeled cDNA molecules 304 can be purified to remove excess barcodes or stochastic barcodes 310. Purification can comprise Ampure bead purification.

As shown in step 2, products from the reverse transcription process in step 1 can be pooled into 1 tube and PCR amplified with a 1^{st} PCR primer pool and a 1^{st} universal PCR primer. Pooling is possible because of the unique label region 314. In particular, the labeled cDNA molecules 304 can be amplified to produce nested PCR labeled amplicons 322. Amplification can comprise multiplex PCR amplification. Amplification can comprise a multiplex PCR amplification with 96 multiplex primers in a single reaction volume. In some embodiments, multiplex PCR amplification can utilize, utilize about, utilize at least, or utilize at most, 10, 20, 40, 50, 70, 80, 90, 10², 10³, 10⁴, 10⁵, 10⁶, 10⁷, 10⁸, 10⁹, 10¹⁰, 10¹¹, 10¹², 10¹³, 10¹⁴, 10¹⁵, 10²⁰, or a number or a range between any of these values, multiplex primers in a single reaction volume. Amplification can comprise using a 1^{st} PCR primer pool 324 comprising custom primers 326A-C targeting specific genes and a universal primer 328. The custom primers 326 can hybridize to a region within the cDNA portion 306′ of the labeled cDNA molecule 304. The universal primer 328 can hybridize to the universal PCR region 316 of the labeled cDNA molecule 304.

As shown in step 3 of FIG. 3, products from PCR amplification in step 2 can be amplified with a nested PCR primers pool and a 2^{nd} universal PCR primer. Nested PCR can minimize PCR amplification bias. In particular, the nested PCR labeled amplicons 322 can be further amplified by nested PCR. The nested PCR can comprise multiplex PCR with nested PCR primers pool 330 of nested PCR primers 332a-c and universal PCR primer 328′ in a single reaction volume. The nested PCR primer pool 328 can contain, contain about, contain at least, or contain at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different nested PCR primers 330. The nested PCR primers 332 can contain an adaptor 334 and hybridize to a region within the cDNA portion 306″ of the labeled amplicon 322. The universal primer 328′ can contain an adaptor 336 and hybridize to the universal PCR region 316 of the labeled amplicon 322. Thus, step 3 produces adaptor-labeled amplicon 338. In some embodiments, nested PCR primers 332 and the 2^{nd} universal PCR primer 328′ may not contain the adaptors 334 and 336. The adaptors 334 and 336 can instead be ligated to the products of nested PCR to produce adaptor-labeled amplicon 338.

As shown in step 4, PCR products from step 3 can be PCR amplified for sequencing using library amplification primers. In particular, the adaptors 334 and 336 can be used to conduct one or more additional assays on the adaptor-labeled amplicon 338. The adaptors 334 and 336 can be hybridized to primers 340 and 342. The one or more primers 340 and 342 can be PCR amplification primers. The one or more primers 340 and 342 can be sequencing primers. The one or more adaptors 334 and 336 can be used for further amplification of the adaptor-labeled amplicons 338. The one or more adaptors 334 and 336 can be used for sequencing the adaptor-labeled amplicon 338. The primer 342 can contain a plate index 344 so that amplicons generated using the same set of barcodes or stochastic barcodes 310 can be sequenced in one sequencing reaction using next generation sequencing (NGS).

### Methods and Compositions for Absolute Quantification of Protein Expression

There are provided, in some embodiments, quantification compositions and methods (e.g., Quanti-CITE-seq) utilizing synthetically modified bead populations with known quantities of a specific protein associated (covalently or absorption bound) with the bead surface to serve as a cell mimic in a single cell multiomics assay. The surface protein on the beads can serve as a protein quantification standard for CITE-seq workflows. The cell mimic (e.g., synthetic protein bead) can be utilized as a cocktail with several unique populations containing known amounts of a specific protein (or multiple) to generate a standard curve across a linear dynamic range. The beads can be mixed directly with suspension cell samples, or run in parallel. If mixed, the cell mimic beads will not have significant impact on cell sample preparation or assay performance in some embodiments.

Protein quantification can be achieved by comparing the CITE-seq readout from protein standard beads provided herein to readout from cell-based samples. Some embodiments disclosed herein provide a plurality of compositions each comprising a cellular component binding reagent (such as a protein binding reagent) that is conjugated with an oligonucleotide, wherein the oligonucleotide comprises a unique identifier for the cellular component binding reagent that it is conjugated with. Cellular component binding reagents (such as barcoded antibodies) and their uses (such as sample indexing of cells) have been described in U.S. Patent Application Publication Nos. US2018/0088112 and US2018/0346970; the content of each of these is incorporated herein by reference in its entirety. CITE-seq workflows can leverage the use of antibody derived tags (antibody conjugated oligonucleotides), and, in some embodiments, the protein standard beads provided herein having previously quantified (e.g., predetermined) average number of specific proteins at surface of each individual bead population can be analyzed by utilizing an antibody-oligonucleotide targeting the specific protein on beads, and having a specific oligonucleotide barcode associated with said antibody (e.g., monoclonal antibody). With a standardized and common oligonucleotide to antibody ratio across an entire CITE-seq library of antibody oligonucleotide conjugates, and a common primer/library prep, the quantification solution can be common across all antibody-oligonucleotides and directly comparable. The CITE-seq “read-out” can provide molecular counts of each specific antibody oligo barcode for every cell in the experiment. In some embodiments, while the oligo barcodes are normalized such that relative quantification of bound antibodies to each target is automatic, the protein standard beads are required to get to absolute quantification of bound antibodies, and to provide a measure of antigen density for every target and cell in the assay.

As described herein, implementing a cell mimic as a protein standard bead within the system, achievable within the same experiment, can control for the current inefficiencies and experimental variables within CITE-seq workflows to provide an quantification of antigen density at the single cell level. In some embodiments the particles provided herein comprise or are derived from Quantibright Beads. Some embodiments provided herein comprise the use of anti-PE AbSeq and a broad cell phenotyping AbSeq cocktail to quantify all the proteins in the CITE-seq panel of a given experiment. Any protein with an associated antibody can be used in the quantification methods and compositions provided herein. Multiple proteins can be immobilized at the surface to better normalize for monoclonal binding variability. In some embodiments the workflow comprises mixing cell-sized beads as cell mimics directly with cell samples in solution prior to sample prep (e.g., AbSeq staining) and the single cell workflow (e.g., Rhapsody). The methods provided herein can comprise using a first particle (e.g., bead) population size of at least, or at most, about 100, 500, 1000, 5000, 10000, 20000, 30000, 40000, 50000, 60000, or 70000, first particles per million cells. In some embodiments, all cellular component binding reagents (e.g., AbSeq) are monoclonal and have a uniform Oligonucleotide: Protein ratio (e.g., 2:1). In some embodiments, a user can quantify quantification molecules on first particles with a standard curve and then be able to quantify all proteins in the CITE-seq experiment. The methods and compositions provided herein (e.g., quantification compositions) can be employed with the same sample, be partitioned in the same cartridge, and undergo the same library preparation, and therefore provide a valuable quantification standard for a user. The methods provided herein can comprise identifying distinct first particle subpopulations based on single protein expression. For example, identifying 4 distinct subpopulations and quantify mean counts, e.g., Low, MedLow, MedHigh, and High. In some embodiments, linear regression correlates quantification molecule per bead to Mean AbSeq Counts (e.g., Log Protein/Bead (x axis) versus Log Mean Counts (y axis)). In some embodiments, the user determines an Absolute Quantified Antigen Density for some or all cellular component targets assayed in an experiment.

In some embodiments, the cellular component target comprises an intracellular protein. In some embodiments, the method comprises fixing and/or permeabilizing cells and/or nuclei before contacting with the cellular component binding reagents described herein. The systems, methods, compositions, and kits provided herein can, in some embodiments, be employed in concert with the systems, methods, compositions, and kits described in PCT Application Publication Nos. WO2024243298A1 (entitled “RNA PROBING AND AMPLIFICATION METHODS FOR SINGLE CELL ANALYSIS ON FIXED CELLS”), WO2023034790A1 (entitled, “USE OF DECOY POLYNUCLEOTIDES IN SINGLE CELL MULTIOMICS”), and WO2023034794A1 (entitled “RNA PRESERVATION AND RECOVERY FROM FIXED CELLS”), the contents of which are incorporated herein by reference in their entireties. The method can comprise: contacting the cells and/or nuclei with a plurality of decoy oligonucleotides capable of hybridizing to at least one of a plurality of non-target nucleic acids in the cells and/or nuclei. In some embodiments, each of the plurality of decoy oligonucleotides are capable of hybridizing to at least a portion of a non-target nucleic acid. In some embodiments, the decoy oligonucleotide: comprises a sequence complementary to at least a portion of a non-target nucleic acid; comprises a sequence identical to or substantially similar to a sequence of the cellular component-binding reagents specific oligonucleotides, optionally the sequence is 3-40 nucleotides in length; has at most 50% sequence identity to the cellular component-binding reagent specific oligonucleotides; does not comprise a UMI; comprises a random sequence, and optionally the random sequence is about four, five, six, seven, eight, nine, ten, eleven, twelve, thirteen, fourteen, or fifteen nucleotides in length; does not comprise any sequence having more than four, five, six, or seven consecutive Ts or As; comprise at least one G or C in every four, five, six, or seven consecutive nucleotides; comprises one or more modified nucleotides; comprises a 5′ modification (e.g., a 5′ Amino Modifier C12 modification (5AmMC12)); comprises a 3′ modification (e.g., a 3′ dideoxy-C modification (ddC)); and/or is 30 to 65 nucleotides in length.

There are provided, in some embodiments, quantification compositions. In some embodiments, the quantification composition comprises: a first particle population comprising two or more first particle subpopulations, and a plurality of quantification molecule-binding reagents. Each first particle of a first particle subpopulation can comprise a predetermined number of copies of a quantification molecule. In some embodiments, the two or more first particle subpopulations differ with respect to the predetermined number of copies of the quantification molecule. Each of the plurality of quantification molecule-binding reagents can comprise a quantification molecule-binding reagent specific oligonucleotide comprising a unique identifier sequence for the quantification molecule. The quantification molecule-binding reagent can be capable of specifically binding to the quantification molecule.

There are provided, in some embodiments, quantification compositions. In some embodiments, the quantification composition comprises: a first particle population comprising two or more first particle subpopulations. Each first particle of a first particle subpopulation can comprise a predetermined number of copies of a quantification molecule. In some embodiments, the two or more first particle subpopulations differ with respect to the predetermined number of copies of the quantification molecule.

The quantification molecule can comprise a protein (e.g., an intracellular protein, an extracellular protein, a cell-surface protein, a cell marker, a B-cell receptor, a T-cell receptor, a major histocompatibility complex, a tumor antigen, a receptor, or any combination thereof). The quantification molecule can comprise a carbohydrate or a lipid. The quantification molecule can comprise a detectable moiety (e.g., phycoerythrin (PE)). The detectable moiety can comprise an optical moiety, a luminescent moiety, a magnetic moiety, an electrochemically active moiety, a nanoparticle, or a combination thereof. The luminescent moiety can comprise a chemiluminescent moiety, an electroluminescent moiety, a photoluminescent moiety, or a combination thereof. The photoluminescent moiety can comprise a fluorescent moiety, a phosphorescent moiety, or a combination thereof. The fluorescent moiety can comprise a fluorescent dye or a fluorescent protein. The quantification molecule can comprise a fluorescent protein selected from the group comprising Sirius, EBFP2, Azurite, mTagBFP2, mKalama1, TagBFP, Aquamarine, SCFPB3A, mTurquoise, mTurquoise2, ECFP, Cerulean, mCerulean3, CyPet, TagCFP, AmCyan, mTFP1, mMidorishi-Cyan, mUKG, TurboGFP, Verdi GFP, Azami Green, ZsGreen, ZsGreen1, TagGFP2, hrGFP, hrGFP2, EGFP, Emerald, mWasabi, Superfolder GFP, Clover, mNeonGreen, Monster Green, Renilla GFP, TurboYFP, TagYFP, EYFP, Topaz, SYFP2, Venus, Citrine, YPet, mPapayal, mKO, E2-Orange, mOrange, mOrange2, mKOk, mKO2, mNectarine, dTomato, tdTomato, DsRed, DsRed2, DsRed-Express, TagRFP, TagRFP-T, mTangerine, DsRed monomer, mApple, mStrawberry, mRuby2, mRuby, Fusion Red, mCherry, mLumin, mRaspberry, TagFP635 (mKate), mKate2, TurboFP635 (Katushka), HcRed-Tandem, mPlum, eqFP650, mNeptune, mCardinal, NirFP (eqFP670), TagRFP675, E2-Crimson, TagRFP657, BphP1-FP, iRFP670, iRFP713/V256C, iRFP682, iRFP702, IFP1.4, IFP2.0, iRFP713 (iRFP), iRFP720, T-Sapphire, mAmetrine, LSS-mOrange, LSS-mKate2, mBeRFP, mKeima Red, LSS-mKate1, or any combination thereof.

The quantification molecules can be present on the surface of the first particle. The quantification molecules present on the surface of the first particle can be conjugated by: (i) a 1,3-dipolar cycloaddition reaction, a hetero-Diels-Alder reaction, a nucleophilic substitution reaction, a non-aldol type carbonyl reaction, an addition to carbon-carbon multiple bond, an oxidation reaction, a click reaction, or any combination thereof; (ii) a reaction between acetylene and azide; and/or (iii) a reaction between an aldehyde or ketone group and a hydrazine or alkoxy amine. The quantification molecule can be absorption bound or covalently bound to the first particle. The quantification molecule can be associated with the first particle via non-covalent interactions (e.g., one or more of ionic bonding, hydrophobic interactions, van der Waals forces, and hydrogen bonding). The quantification molecule can be conjugated to the first particle by a 1,3-dipolar cycloaddition reaction, a hetero-Diels-Alder reaction, a nucleophilic substitution reaction, a non-aldol type carbonyl reaction, an addition to carbon-carbon multiple bond, an oxidation reaction, a click reaction, or any combination thereof. The quantification molecule can comprise a first ligand and the first particle can comprise a second ligand, the first ligand and the second ligand can be a specific binding pair, and the specific binding pair can comprise a first member of a specific binding pair and a second member of a specific binding pair that bind one another with high affinity, high avidity, and/or high specificity. In some embodiments, the binding between the first and second member of the specific binding pair occurs via covalent bonding; the binding between the first and second member of the specific binding pair occurs via non-covalent interactions; the binding between the first and the second member of the specific binding pair has a dissociation constant Ka between about 10-10 to about 10-15 mol/L; and/or the first and the second member of the specific binding pair is each selected from the group comprising an antibody or an antigen-binding portion thereof and an antigen, an biotin moiety and an avidin moiety, a dinitrophenol (DNP) and an anti-DNP antibody, a digoxin and an anti-digoxin antibody, a digoxigenin and an anti-digoxigenin antibody, a hapten and an anti-hapten, a polysaccharide and a polysaccharide binding moiety, a lectin and a receptor, a ligand and a receptor, a fluorescein and an anti-fluorescein antibody, complementary nucleic acids, derivatives therefore, and fragments thereof.

The first particle can comprise a bead. In some embodiments, the bead comprises: a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof; a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof; and/or a disruptable hydrogel particle. The first particle can comprise a diameter of about 10 μm to about 100 μm (e.g., about 35 μm). The first particle can be configured to have the dimensions of a mammalian cell (e.g., a human cell). The first particle population can comprise at least 2, 3, 4, 5, 6, 7, 8, 9, or 10 first particle subpopulations. Each first particle subpopulation can comprise at least about 2, 5, 10, 50, 100, 200, 500, 750, 1000, 2500, 5000, 7500, or 10000, first particles. In some embodiments, a first particle of a first particle subpopulation can comprise at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, or 10000, quantification molecules. The quantification molecule-binding reagent specific oligonucleotide can comprise (i) a second molecular label and/or (ii) a second universal sequence. At least ten of the plurality of quantification molecule-binding reagent specific oligonucleotides can comprise different second molecular label sequences. The quantification molecule-binding reagents can comprise a protein, an antibody, an aptamer, a tetramer, a protein scaffold, or a combination thereof. The aptamer and the reagent oligonucleotide can be a single polynucleotide.

The quantification molecule-binding reagents can comprise an antibody or fragment thereof. The antibody or fragment thereof can comprise a monoclonal antibody, a Fab, a Fab′, a F(ab′)², a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. Each first particle of a first particle subpopulation can comprise a predetermined number of copies of one or more secondary quantification molecules. In some embodiments, the two or more first particle subpopulations differ with respect to the predetermined number of copies of said one or more secondary quantification molecules. The quantification composition can comprise a second particle population comprising two or more second particle subpopulations. Each second particle of a second particle subpopulation can comprise a predetermined number of copies of one or more secondary quantification molecules. In some embodiments, the two or more first particle subpopulations differ with respect to the predetermined number of copies of one or more secondary quantification molecules. The quantification composition can comprise a plurality of secondary quantification molecule-binding reagents. Each of the plurality of secondary quantification molecule-binding reagents can comprise a secondary quantification molecule-binding reagent specific oligonucleotide comprising a unique identifier sequence for the secondary quantification molecule, and the secondary quantification molecule-binding reagent can be capable of specifically binding to at least one of the one or more secondary quantification molecules.

Each first particle of a first particle subpopulation can comprise first particle specific oligonucleotide(s) comprising a unique identifier sequence for the subpopulation. At least one of the first particle specific oligonucleotide(s) can be immobilized on, partially immobilized, enclosed in, or partially enclosed in the first particle. The first particle specific oligonucleotide(s) can be conjugated to the first particle. The first particle specific oligonucleotide(s) can be conjugated to the first particle through a chemical group selected from the group consisting of a UV photocleavable group, a streptavidin, a biotin, an amine, and a combination thereof. The first particle specific oligonucleotide(s) can comprise a sequence complementary to a capture sequence configured to capture the first particle specific oligonucleotide. The sequence complementary to the capture sequence can comprise a poly(dA) region. Each of the first particle specific oligonucleotide(s) can be 10 to 500 nucleotides in length. Each of the first particle specific oligonucleotide(s) can comprise a linker functional group. The first particle can comprise a solid support functional group. The support functional group and the linker functional group can be associated with each other. The linker functional group and the support functional group can be individually selected from the group consisting of C6, biotin, streptavidin, primary amine(s), aldehyde(s), ketone(s), and any combination thereof.

There are provided, in some embodiments, methods for cellular component quantification. In some embodiments, the method comprises: contacting one or more cells with a plurality of cellular component-binding reagents, wherein the one or more cells comprises a plurality of cellular component targets, wherein each of the plurality of cellular component-binding reagents comprises a cellular component-binding reagent specific oligonucleotide comprising a unique identifier sequence for the cellular component-binding reagent, and wherein the cellular component-binding reagent is capable of specifically binding to at least one of the plurality of cellular component targets. The method can comprise contacting a quantification composition disclosed herein with a plurality of quantification molecule-binding reagents, wherein each of the plurality of quantification molecule-binding reagents comprises a quantification molecule-binding reagent specific oligonucleotide comprising a unique identifier sequence for the quantification molecule, and wherein the quantification molecule-binding reagent is capable of specifically binding to the quantification molecule. The method can comprise barcoding the cellular component-binding reagent specific oligonucleotides with a plurality of oligonucleotide barcodes to generate a plurality of barcoded cellular component-binding reagent specific oligonucleotides each comprising a sequence complementary to at least a portion of the unique identifier sequence and a molecular label sequence. The method can comprise barcoding the quantification molecule-binding reagent specific oligonucleotides with a plurality of oligonucleotide barcodes to generate a plurality of barcoded quantification molecule-binding reagent specific oligonucleotides each comprising a sequence complementary to at least a portion of the unique identifier sequence and a molecular label sequence. The method can comprise obtaining sequencing data comprising a plurality of sequencing reads of the plurality of barcoded cellular component-binding reagent specific oligonucleotides, or products thereof, wherein each of the plurality of sequencing reads comprises a molecular label sequence and at least a portion of the unique identifier sequence. In some embodiments, the number of unique molecular label sequences associated with the unique identifier sequence for the cellular component-binding reagent capable of specifically binding to the at least one cellular component target in the sequencing data indicates a pre-adjusted number of copies of the at least one cellular component target in the one or more cells. The method can comprise obtaining sequencing data comprising a plurality of sequencing reads of the plurality of barcoded quantification molecule-binding reagent specific oligonucleotides, or products thereof, wherein each of the plurality of sequencing reads comprises a molecular label sequence and at least a portion of the unique identifier sequence. In some embodiments, the number of unique molecular label sequences associated with the unique identifier sequence for the quantification molecule-binding reagent in the sequencing data indicates the experimental number of copies of the quantification molecule in each first particle subpopulation.

The method can comprise generating a correction factor by comparing the experimental number of copies of the quantification molecule in each first particle subpopulation with the predetermined number of copies of the quantification molecule in each first particle subpopulation. The method can comprise adjusting the pre-adjusted number of copies of the at least one cellular component target in the one or more cells with the correction factor to generate an adjusted number of copies of the at least one cellular component target in the one or more cells. In some embodiments, generating the correction value can comprise generating a calibration curve. The method can comprise contacting the quantification composition with the one or more cells, and the step of contacting one or more cells with a plurality of cellular component-binding reagents can comprise contacting the quantification composition with the plurality of quantification molecule-binding reagents. The step of contacting one or more cells with the plurality of cellular component-binding reagents and the step of contacting the quantification composition with the plurality of quantification molecule-binding reagents can be performed separately. The plurality of cellular component-binding reagents can comprise at least 10 cellular component-binding reagents each capable of specifically binding to at least one of at least 10 cellular component targets. The cellular component target can comprise an intracellular protein, a carbohydrate, a lipid, a protein, an extracellular protein, a cell marker, a B-cell receptor, a T-cell receptor, a major histocompatibility complex, a tumor antigen, a receptor, or any combination thereof. In some embodiments, the at least one cellular component target does not comprise the quantification molecule or the one or more secondary quantification molecules. The at least one cellular component target can comprise the quantification molecule or the one or more secondary quantification molecules. In some embodiments, at least one of the quantification molecule-binding reagents and the cellular component-binding reagents can be the same. The quantification molecule-binding reagents and the cellular component-binding reagents can comprise a protein, an antibody, an aptamer, a tetramer, a protein scaffold, or a combination thereof. In some embodiments, the aptamer and the reagent oligonucleotide is a single polynucleotide.

The quantification molecule-binding reagents and the cellular component-binding reagents can comprise an antibody or fragment thereof. The antibody or fragment thereof can comprise a monoclonal antibody, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, the quantification molecule-binding reagents and cellular component-binding reagents have the same clonotype. The method can comprise: after contacting the plurality of quantification molecule-binding reagents with the one or more cells, removing one or more quantification molecule-binding reagents of the plurality of quantification molecule-binding reagents that are not contacted with the first particle population. The method can comprise: removing the one or more quantification molecule-binding reagents not contacted with the first particle population comprises: removing the one or more quantification molecule-binding reagents not contacted with the quantification molecules. The method can comprise: after contacting the plurality of cellular component-binding reagents with the one or more cells, removing one or more cellular component-binding reagents of the plurality of cellular component-binding reagents that are not contacted with the one or more cells. In some embodiments, removing the one or more cellular component-binding reagents not contacted with the one or more cells comprises: removing the one or more cellular component-binding reagents not contacted with the respective at least one of the plurality of cellular component targets.

The one or more cells can comprise a plurality of single cells. The plurality of single cells can be derived from one or more samples (e.g., a plurality of samples). The one or more cells can comprise a plurality of cells, a plurality of single cells, a tissue, a tumor sample, or any combination thereof. The sample can comprise peripheral blood mononuclear cells or immune cells. The immune cells can comprise B cells, T cells or a combination thereof. In some embodiments, the contacting step comprises: partitioning the plurality of single cells, the first particle population, and a pool of solid supports to a plurality of partitions, wherein a partition of the plurality of partitions comprises a single cell and a single solid support, and wherein a partition of the plurality of partitions comprises a single first particle and a single solid support. In some embodiments, partition can be a well or droplet. The method can comprise lysing each cell in the partitions. The method can comprise pooling the solid supports from said partitions.

The plurality of oligonucleotide barcodes can be associated with a solid support. Each oligonucleotide barcode can comprise a first universal sequence, a first molecular label, and a target-binding region capable of hybridizing to one or more of a nucleic acid target, a cellular component-binding reagent specific oligonucleotide, a quantification molecule-binding reagent specific oligonucleotide, and a sample indexing oligonucleotide. At least 10 of the plurality of oligonucleotide barcodes can comprise different first molecular label sequences. The plurality of oligonucleotide barcodes each can comprise a cell label. Each cell label of the plurality of oligonucleotide barcodes can comprise at least 6 nucleotides. Oligonucleotide barcodes associated with the same solid support can comprise the same cell label. Oligonucleotide barcodes associated with different solid supports can comprise different cell labels. The cellular component-binding reagent specific oligonucleotide can comprise one or more of: a second molecular label sequence; an alignment sequence adjacent to a poly(dA) region; a linker; a sequence complementary to a capture sequence configured to capture the cellular component-binding reagent specific oligonucleotide; and a second universal sequence. At least ten of the plurality of cellular component-binding reagent specific oligonucleotides can comprise different second molecular label sequences. The second molecular label sequences of at least two cellular component-binding reagent specific oligonucleotides can be different, and the unique identifier sequences of the at least two cellular component-binding reagent specific oligonucleotides can be identical. The second molecular label sequences of at least two cellular component-binding reagent specific oligonucleotides can be different, and the unique identifier sequences of the at least two cellular component-binding reagent specific oligonucleotides can be different. The alignment sequence can be one or more nucleotides, or two or more nucleotides, in length. The sequence complementary to the capture sequence can comprise a poly(dA) region. The cellular component-binding reagent specific oligonucleotide can be associated with the first cellular component-binding reagent through the linker.

The method can comprise: contacting the quantification composition disclosed herein with a plurality of secondary quantification molecule-binding reagents, wherein each of the plurality of secondary quantification molecule-binding reagents comprises a secondary quantification molecule-binding reagent specific oligonucleotide comprising a unique identifier sequence for the secondary quantification molecule, and wherein the secondary quantification molecule-binding reagent is capable of specifically binding to at least one of the one or more secondary quantification molecules. The method can comprise: barcoding the secondary quantification molecule-binding reagent specific oligonucleotides with a plurality of oligonucleotide barcodes to generate a plurality of barcoded secondary quantification molecule-binding reagent specific oligonucleotides each comprising a sequence complementary to at least a portion of the unique identifier sequence and a molecular label sequence. The method can comprise: obtaining sequencing data comprising a plurality of sequencing reads of the plurality of barcoded secondary quantification molecule-binding reagent specific oligonucleotides, or products thereof, wherein each of the plurality of sequencing reads comprises a molecular label sequence and at least a portion of the unique identifier sequence. In some embodiments, the number of unique molecular label sequences associated with the unique identifier sequence for the secondary quantification molecule-binding reagent in the sequencing data indicates the experimental number of copies of the secondary quantification molecule in each first particle subpopulation or second particle subpopulation.

Generating the correction factor can comprise comparing the experimental number of copies of the secondary quantification molecule in each first particle subpopulation with the predetermined number of copies of the secondary quantification molecule in each first particle subpopulation. Generating the correction factor can comprise comparing the experimental number of copies of the secondary quantification molecule in each second particle subpopulation with the predetermined number of copies of the secondary quantification molecule in each second particle subpopulation. The number of unique molecular label sequences can comprise the number of unique first molecular label sequences, the number of unique second molecular label sequences, or a combination thereof. In some embodiments, the method comprises: barcoding the first particle specific oligonucleotides with a plurality of oligonucleotide barcodes to generate a plurality of barcoded first particle specific oligonucleotides each comprising a sequence complementary to at least a portion of the unique identifier sequence and a molecular label sequence; obtaining sequencing data comprising a plurality of sequencing reads of the plurality of barcoded first particle specific oligonucleotides, or products thereof, wherein each of the plurality of sequencing reads comprises at least a portion of the unique identifier sequence and the cell label sequence; and associating the plurality of sequencing reads of the plurality of barcoded quantification molecule-binding reagent specific oligonucleotides, or products thereof, with a first particle subpopulation based on a shared cell label sequence.

In some embodiments, the one or more cells comprises copies of a nucleic acid target. The method can comprise: contacting the plurality of oligonucleotide barcodes with the copies of the nucleic acid target for hybridization; extending the plurality of oligonucleotide barcodes hybridized to the copies of a nucleic acid target to generate a plurality of barcoded nucleic acid molecules each comprising a sequence complementary to at least a portion of the nucleic acid target; and obtaining sequencing data of the plurality of barcoded nucleic acid molecules, or products thereof, to determine the copy number of the nucleic acid target in the one or more single cells. Extending the plurality of oligonucleotide barcodes hybridized to the copies of the nucleic acid target can comprise extending the plurality of oligonucleotide barcodes hybridized to the copies of the nucleic acid target using a reverse transcriptase (e.g., a viral reverse transcriptase). The method can comprise determining the copy number of the nucleic acid target in the one or more cells based on the number of first molecular labels with distinct sequences associated with the plurality of barcoded nucleic acid molecules, or products thereof. The sequence of the nucleic acid target in the plurality of barcoded nucleic acid molecules can comprise a subsequence of the nucleic acid target. Determining the copy number of the nucleic acid target can comprise determining the copy number of each of a plurality of nucleic acid targets in the one or more cells based on the number of molecular labels with distinct sequences associated with the plurality of barcoded nucleic acid molecules, or products thereof, comprising a sequence of the each of the plurality of nucleic acid targets. The sequence of the each of the plurality of nucleic acid targets can comprise a subsequence of the each of the plurality of nucleic acid targets.

Barcoding the cellular component-binding reagent specific oligonucleotides can comprise contacting with the plurality of oligonucleotide barcode for hybridization and extending the plurality of oligonucleotide barcodes hybridized to the cellular component-binding reagent specific oligonucleotides to generate the plurality of barcoded cellular component-binding reagent specific oligonucleotides. Barcoding the quantification molecule-binding reagent specific oligonucleotides can comprise contacting with the plurality of oligonucleotide barcode for hybridization and extending the plurality of oligonucleotide barcodes hybridized to the quantification molecule-binding reagent specific oligonucleotides to generate the plurality of barcoded quantification molecule-binding reagent specific oligonucleotides. Barcoding the secondary quantification molecule-binding reagent specific oligonucleotides can comprise contacting with the plurality of oligonucleotide barcode for hybridization and extending the plurality of oligonucleotide barcodes hybridized to the secondary quantification molecule-binding reagent specific oligonucleotides to generate the plurality of barcoded secondary quantification molecule-binding reagent specific oligonucleotides. Barcoding the first particle specific oligonucleotides can comprise contacting with the plurality of oligonucleotide barcode for hybridization and extending the plurality of oligonucleotide barcodes hybridized to the first particle specific oligonucleotides to generate the plurality of barcoded first particle specific oligonucleotides.

Barcoding the cellular component-binding reagent specific oligonucleotides can comprise contacting with the plurality of oligonucleotide barcode for hybridization and extending the cellular component-binding reagent specific oligonucleotides hybridized to the plurality of oligonucleotide barcodes to generate the plurality of barcoded cellular component-binding reagent specific oligonucleotides. Barcoding the quantification molecule-binding reagent specific oligonucleotides can comprise contacting with the plurality of oligonucleotide barcode for hybridization and extending the quantification molecule-binding reagent specific oligonucleotides hybridized to the plurality of oligonucleotide barcodes to generate the plurality of barcoded quantification molecule-binding reagent specific oligonucleotides. Barcoding the secondary quantification molecule-binding reagent specific oligonucleotides can comprise contacting with the plurality of oligonucleotide barcode for hybridization and extending the secondary quantification molecule-binding reagent specific oligonucleotides hybridized to the plurality of oligonucleotide barcodes to generate the plurality of barcoded secondary quantification molecule-binding reagent specific oligonucleotides. Barcoding the first particle specific oligonucleotides can comprise contacting with the plurality of oligonucleotide barcode for hybridization and extending the first particle specific oligonucleotides hybridized to the plurality of oligonucleotide barcodes to generate the plurality of barcoded first particle specific oligonucleotides. Obtaining sequencing data of the plurality of barcoded cellular component-binding reagent specific oligonucleotides, or products thereof, can comprise attaching the binding sites of sequencing primers and/or sequencing adaptors to the plurality of barcoded cellular component-binding reagent specific oligonucleotides, or products thereof.

Obtaining sequencing data of the plurality of barcoded quantification molecule-binding reagent specific oligonucleotides, or products thereof, can comprise attaching the binding sites of sequencing primers and/or sequencing adaptors to the plurality of barcoded quantification molecule-binding reagent specific oligonucleotides, or products thereof. Obtaining sequencing data of the plurality of barcoded secondary quantification molecule-binding reagent specific oligonucleotides, or products thereof, can comprise attaching the binding sites of sequencing primers and/or sequencing adaptors to the plurality of barcoded secondary quantification molecule-binding reagent specific oligonucleotides, or products thereof. Obtaining sequencing data of the plurality of barcoded first particle specific oligonucleotides, or products thereof, can comprise attaching the binding sites of sequencing primers and/or sequencing adaptors to the plurality of barcoded first particle specific oligonucleotides, or products thereof. Obtaining sequencing data of the plurality of barcoded nucleic acid molecules, or products thereof, can comprise attaching the binding sites of sequencing primers and/or sequencing adaptors to the plurality of barcoded nucleic acid molecules, or products thereof.

The first universal sequence and the second universal sequence can be the same. The first universal sequence and the second universal sequence can be different. The first universal sequence and/or the second universal sequence can comprise the binding sites of sequencing primers and/or sequencing adaptors, complementary sequences thereof, and/or portions thereof. The sequencing adaptors can comprise a P5 sequence, a P7 sequence, complementary sequences thereof, and/or portions thereof. The sequencing primers can comprise a Read 1 sequencing primer, a Read 2 sequencing primer, complementary sequences thereof, and/or portions thereof.

The method can comprise contacting each of the plurality of samples with a sample indexing composition of a plurality of sample indexing compositions, respectively. In some embodiments, each of the plurality of samples comprises one or more cells each comprising one or more cellular component targets, wherein the sample indexing composition comprises a cellular component-binding reagent associated with a sample indexing oligonucleotide, wherein the cellular component-binding reagent is capable of specifically binding to at least one of the one or more cellular component targets. The sample indexing oligonucleotide can comprise a sample indexing sequence, or a precursor thereof, and wherein sample indexing sequences of at least two sample indexing compositions of the plurality of sample indexing compositions comprise different sequences. The method can comprise: identifying sample origin of at least one cell of the one or more cells based on the sample indexing sequence of at least one sample indexing oligonucleotide of the plurality of sample indexing compositions. In some embodiments, identifying the sample origin of the at least one cell comprises: barcoding sample indexing oligonucleotides of the plurality of sample indexing compositions using the plurality of oligonucleotide barcodes to generate a plurality of barcoded sample indexing oligonucleotides; obtaining sequencing data of the plurality of barcoded sample indexing oligonucleotides, or products thereof; and identifying the sample origin of the cell based on the sample indexing sequence of at least one barcoded sample indexing oligonucleotide of the plurality of barcoded sample indexing oligonucleotides in the sequencing data. Identifying the sample origin of the at least one cell can comprise identifying the presence or absence of the sample indexing sequence of at least one sample indexing oligonucleotide, or of at least one barcoded sample indexing oligonucleotide, in the sequencing data.

The target-binding region can comprise an oligo dT sequence, a random sequence, a target-specific sequence, or a combination thereof. The target-binding region can comprise a poly(dT) region, and wherein the nucleic acid target can comprise a poly(dA) region. The solid support can comprise a synthetic particle. The solid support can comprise a planar surface. At least one of the plurality of oligonucleotide barcodes can be immobilized on, partially immobilized, enclosed in, or partially enclosed in the synthetic particle. The synthetic particle can be disruptable. The synthetic particle can comprise a bead. The bead can comprise a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof. The synthetic particle can comprise a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof. The synthetic particle can comprise a disruptable hydrogel particle. Each of the plurality of oligonucleotide barcodes can comprise a linker functional group. The synthetic particle can comprise a solid support functional group. The support functional group and the linker functional group can be associated with each other. The linker functional group and the support functional group can be individually selected from the group consisting of C6, biotin, streptavidin, primary amine(s), aldehyde(s), ketone(s), and any combination thereof.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-90. (canceled)

91. A method of cellular component quantification, comprising:
contacting one or more cells with a plurality of cellular component-binding reagents, wherein the one or more cells comprise a plurality of cellular component targets, wherein each of the plurality of cellular component-binding reagents comprises a cellular component-binding reagent specific oligonucleotide comprising a unique identifier sequence for the cellular component-binding reagent, and wherein the cellular component-binding reagent is capable of specifically binding to at least one of the plurality of cellular component targets;
contacting a quantification composition with a plurality of quantification molecule-binding reagents,
wherein the quantification composition comprises a first particle population comprising two or more first particle subpopulations, wherein each first particle of a first particle subpopulation comprises a predetermined number of copies of a quantification molecule,
wherein the two or more first particle subpopulations differ with respect to the predetermined number of copies of the quantification molecule,
wherein each of the plurality of quantification molecule-binding reagents comprises a quantification molecule-binding reagent specific oligonucleotide comprising a unique identifier sequence for the quantification molecule, and
wherein the quantification molecule-binding reagent is capable of specifically binding to the quantification molecule;
barcoding the cellular component-binding reagent specific oligonucleotides with a plurality of oligonucleotide barcodes to generate a plurality of barcoded cellular component-binding reagent specific oligonucleotides each comprising a sequence complementary to at least a portion of the unique identifier sequence and a molecular label sequence;
barcoding the quantification molecule-binding reagent specific oligonucleotides with a plurality of oligonucleotide barcodes to generate a plurality of barcoded quantification molecule-binding reagent specific oligonucleotides each comprising a sequence complementary to at least a portion of the unique identifier sequence and a molecular label sequence;
obtaining sequencing data comprising a plurality of sequencing reads of the plurality of barcoded cellular component-binding reagent specific oligonucleotides, or products thereof, wherein each of the plurality of sequencing reads comprises a molecular label sequence and at least a portion of the unique identifier sequence,
wherein the number of unique molecular label sequences associated with the unique identifier sequence for the cellular component-binding reagent capable of specifically binding to the at least one cellular component target in the sequencing data indicates a pre-adjusted number of copies of the at least one cellular component target in the one or more cells;
obtaining sequencing data comprising a plurality of sequencing reads of the plurality of barcoded quantification molecule-binding reagent specific oligonucleotides, or products thereof, wherein each of the plurality of sequencing reads comprises a molecular label sequence and at least a portion of the unique identifier sequence,
wherein the number of unique molecular label sequences associated with the unique identifier sequence for the quantification molecule-binding reagent in the sequencing data indicates the experimental number of copies of the quantification molecule in each first particle subpopulation;
generating a correction factor by comparing the experimental number of copies of the quantification molecule in each first particle subpopulation with the predetermined number of copies of the quantification molecule in each first particle subpopulation; and
adjusting the pre-adjusted number of copies of the at least one cellular component target in the one or more cells with the correction factor to generate an adjusted number of copies of the at least one cellular component target in the one or more cells.

92. The method of claim 91, wherein generating the correction factor comprises generating a calibration curve.

93. The method of claim 91, wherein the step of contacting the one or more cells with the plurality of cellular component-binding reagents and the step of contacting the quantification composition with the plurality of quantification molecule-binding reagents are performed together in a common reaction mixture and/or separately in different reaction mixtures.

94. The method of claim 91, wherein:
the plurality of cellular component-binding reagents comprises at least 10 cellular component-binding reagents each capable of specifically binding to at least one of at least 10 cellular component targets; and/or the cellular component target comprises an intracellular protein, a carbohydrate, a lipid, a protein, an extracellular protein, a cell marker, a B-cell receptor, a T-cell receptor, a major histocompatibility complex, a tumor antigen, a receptor, or any combination thereof.

95. The method of claim 91, wherein:
at least one cellular component target does not comprise the quantification molecule;
at least one cellular component target comprises the quantification molecule;
and/or at least one of the quantification molecule-binding reagents and the cellular component-binding reagents are the same.

96. The method of claim 91, wherein:
the quantification molecule-binding reagents and the cellular component-binding reagents comprise an antibody or fragment thereof, a monoclonal antibody, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof; and/or the quantification molecule-binding reagents and cellular component-binding reagents have the same clonotype.

97. The method of claim 91, wherein the one or more cells comprise a plurality of single cells, and wherein the contacting step comprises: partitioning the plurality of single cells, the first particle population, and a pool of solid supports to a plurality of partitions, wherein a partition of the plurality of partitions comprises a single cell and a single solid support, and wherein a partition of the plurality of partitions comprises a single first particle and a single solid support.

98. The method of claim 97, wherein:
the partition is a well or droplet,
the method comprises lysing each cell in the partitions, and
the method comprises pooling the solid supports from said partitions.

99. The method of claim 97, wherein the plurality of oligonucleotide barcodes are associated with a solid support,
wherein each oligonucleotide barcode comprises a first universal sequence, a first molecular label, and a target-binding region capable of hybridizing to one or more of a nucleic acid target, a cellular component-binding reagent specific oligonucleotide, and a quantification molecule-binding reagent specific oligonucleotide, wherein at least 10 of the plurality of oligonucleotide barcodes comprise different first molecular label sequences,
wherein the plurality of oligonucleotide barcodes each comprise a cell label,
wherein oligonucleotide barcodes associated with the same solid support comprise the same cell label, and
wherein oligonucleotide barcodes associated with different solid supports comprise different cell labels.

100. The method of claim 91, wherein the cellular component-binding reagent specific oligonucleotide comprises one or more of:
a second molecular label sequence, and wherein:
the second molecular label sequences of at least two cellular component-binding reagent specific oligonucleotides are different, and wherein the unique identifier sequences of the at least two cellular component-binding reagent specific oligonucleotides are identical; and/or
the second molecular label sequences of at least two cellular component-binding reagent specific oligonucleotides are different, and wherein the unique identifier sequences of the at least two cellular component-binding reagent specific oligonucleotides are different;
a linker, wherein the cellular component-binding reagent specific oligonucleotide is associated with the cellular component-binding reagent through the linker;
a sequence complementary to a capture sequence configured to capture the cellular component-binding reagent specific oligonucleotide;
a poly(dA) region; and/or
a second universal sequence.

101. The method of claim 91, wherein:
the first particle comprises a diameter of about 10 μm to about 100 μm;
the first particle comprises a diameter of about 35 μm; and/or the first particle is configured to have the dimensions of a mammalian cell.

102. The method of claim 91, wherein:
the first particle population comprises at least 2, 3, 4, 5, 6, 7, 8, 9, or 10 first particle subpopulations;
each first particle subpopulation comprises at least about 2, 5, 10, 50, 100, 200, 500, 750, 1000, 2500, 5000, 7500, or 10000, first particles; and/or
a first particle of a first particle subpopulation comprises at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, or 10000, quantification molecules.

103. The method of claim 91,
wherein each first particle of a first particle subpopulation comprises a predetermined number of copies of one or more secondary quantification molecules, and
wherein the two or more first particle subpopulations differ with respect to the predetermined number of copies of said one or more secondary quantification molecules.

104. The method of claim 103, wherein the method comprises:
contacting the quantification composition with a plurality of secondary quantification molecule-binding reagents, wherein each of the plurality of secondary quantification molecule-binding reagents comprises a secondary quantification molecule-binding reagent specific oligonucleotide comprising a unique identifier sequence for the secondary quantification molecule, and wherein the secondary quantification molecule-binding reagent is capable of specifically binding to at least one of the one or more secondary quantification molecules; and
barcoding the secondary quantification molecule-binding reagent specific oligonucleotides with a plurality of oligonucleotide barcodes to generate a plurality of barcoded secondary quantification molecule-binding reagent specific oligonucleotides each comprising a sequence complementary to at least a portion of the unique identifier sequence and a molecular label sequence;
obtaining sequencing data comprising a plurality of sequencing reads of the plurality of barcoded secondary quantification molecule-binding reagent specific oligonucleotides, or products thereof, wherein each of the plurality of sequencing reads comprises a molecular label sequence and at least a portion of the unique identifier sequence,
wherein the number of unique molecular label sequences associated with the unique identifier sequence for the secondary quantification molecule-binding reagent in the sequencing data indicates the experimental number of copies of the secondary quantification molecule in each first particle subpopulation, and
wherein generating the correction factor comprises comparing the experimental number of copies of the secondary quantification molecule in each first particle subpopulation with the predetermined number of copies of the secondary quantification molecule in each first particle subpopulation.

105. The method of claim 91, wherein the quantification composition further comprises:
a second particle population comprising two or more second particle subpopulations,
wherein each second particle of a second particle subpopulation comprises a predetermined number of copies of one or more secondary quantification molecules, and
wherein the two or more second particle subpopulations differ with respect to the predetermined number of copies of one or more secondary quantification molecules.

106. The method of claim 105, wherein the method comprises:
contacting the quantification composition with a plurality of secondary quantification molecule-binding reagents, wherein each of the plurality of secondary quantification molecule-binding reagents comprises a secondary quantification molecule-binding reagent specific oligonucleotide comprising a unique identifier sequence for the secondary quantification molecule, and wherein the secondary quantification molecule-binding reagent is capable of specifically binding to at least one of the one or more secondary quantification molecules;
barcoding the secondary quantification molecule-binding reagent specific oligonucleotides with a plurality of oligonucleotide barcodes to generate a plurality of barcoded secondary quantification molecule-binding reagent specific oligonucleotides each comprising a sequence complementary to at least a portion of the unique identifier sequence and a molecular label sequence; and
obtaining sequencing data comprising a plurality of sequencing reads of the plurality of barcoded secondary quantification molecule-binding reagent specific oligonucleotides, or products thereof, wherein each of the plurality of sequencing reads comprises a molecular label sequence and at least a portion of the unique identifier sequence,
wherein the number of unique molecular label sequences associated with the unique identifier sequence for the secondary quantification molecule-binding reagent in the sequencing data indicates the experimental number of copies of the secondary quantification molecule in each second particle subpopulation, and
wherein generating the correction factor comprises comparing the experimental number of copies of the secondary quantification molecule in each second particle subpopulation with the predetermined number of copies of the secondary quantification molecule in each second particle subpopulation.

107. The method of claim 99, wherein each first particle of a first particle subpopulation comprises first particle specific oligonucleotide(s) comprising a unique identifier sequence for the subpopulation, and wherein the method comprises:
barcoding the first particle specific oligonucleotides with a plurality of oligonucleotide barcodes to generate a plurality of barcoded first particle specific oligonucleotides each comprising a sequence complementary to at least a portion of the unique identifier sequence and a molecular label sequence;
obtaining sequencing data comprising a plurality of sequencing reads of the plurality of barcoded first particle specific oligonucleotides, or products thereof, wherein each of the plurality of sequencing reads comprises at least a portion of the unique identifier sequence and the cell label sequence; and
associating the plurality of sequencing reads of the plurality of barcoded quantification molecule-binding reagent specific oligonucleotides, or products thereof, with a first particle subpopulation based on a shared cell label sequence.

108. The method of claim 91, wherein the one or more cells comprise one or more single cells comprising copies of a nucleic acid target, further comprising:
contacting the plurality of oligonucleotide barcodes with the copies of the nucleic acid target for hybridization, extending the plurality of oligonucleotide barcodes hybridized to the copies of a nucleic acid target to generate a plurality of barcoded nucleic acid molecules each comprising a sequence complementary to at least a portion of the nucleic acid target; and
obtaining sequencing data of the plurality of barcoded nucleic acid molecules, or products thereof, to determine the copy number of the nucleic acid target in the one or more single cells, wherein the copy number of the nucleic acid target is determined based on a number of molecular labels with distinct sequences associated with the plurality of barcoded nucleic acid molecules, or products thereof.

109. A quantification composition, comprising:
a first particle population comprising two or more first particle subpopulations,
wherein each first particle of a first particle subpopulation comprises a predetermined number of copies of a quantification molecule, and
wherein the two or more first particle subpopulations differ with respect to the predetermined number of copies of the quantification molecule; and
a plurality of quantification molecule-binding reagents,
wherein each of the plurality of quantification molecule-binding reagents comprises a quantification molecule-binding reagent specific oligonucleotide comprising a unique identifier sequence for the quantification molecule, and
wherein the quantification molecule-binding reagent is capable of specifically binding to the quantification molecule.

110. A quantification composition, comprising:
a first particle population comprising two or more first particle subpopulations,
wherein each first particle of a first particle subpopulation comprises a predetermined number of copies of a quantification molecule, and
wherein the two or more first particle subpopulations differ with respect to the predetermined number of copies of the quantification molecule.
